SEQUENCE LISTING (36 sequences)

SEQ ID NO: 1 - DNA, 2013 bp, Feline calicivirus
  CDS (1)..(2013)
atgtgctcaacctgcgctaacgtgcttaaatattatgattgggacccccatttcaaattggtaatcaaccccaacaacttcctctctgttggcttttgtagtaaccctttaatgtgttgctacccagaactccttccggaatttggaactgtttgggattgcgatcggtcaccacttgaaatttacctagaatcaatacttggtgatgatgaatgggcatccacttttgacgctgttgacccagtcgttcccccaatgcactggggtgctgctggaaaaattttccagccacaccccggtgttctcatgcaccatctcattggtaaggttgctgcaggttgggaccccgatctgcctctaattcgactcgaggcggatgacgggtcaatcacagcacccgagcaaggaacaatggttggcggcgtcatcgctgaacccagcgcccagatgtcaacagctgctgatatggccaccgggaaaagcgttgattctgagtgggaggcattcttctcctttcacaccagcgtcaattggagtacatctgaaacccaaggaaagattctcttcaaacaatccttaggccctttgctcaacccatatctagaacaccttgctaagctatatgttgcgtggtctgggtcgattgaggttaggttctctatctctggctctggtgtctttggtgggaagctcgcagctattgttgtacctcctggggttgatccagtgcagagtacttcgatgctacaatacccccatgttttgtttgatgctcgtcaggtggaaccagttatcttctgtcttcctgatctaagaagcaccctgtaccaccttatgtctgacactgacactacatccttggtcattatggtgtacaatgatctcatcaatccctatgccaatgatgccaactcttctgggtgtattgtcactgtcgagacaaaacctggccctgacttcaagtttcacctccttaagccacccggatctatgctaacccatggctctatcccttctgatttaattcccaaaacatcttcgctctggatcggtaaccgctactggtcagacataactgattttgtgattcggccgtttgtcttccaagcaaatcgtcattttgactttaatcaagagaccgcagggtggagcacaccacggtttcggcctatatctgttaccattactgaacagaacggagcaaaattgggcattggggtggcaacagattacatagtgcctggaatccctgatggctggcctgacaccacaattcctggggagttgataccagctggtgattacgcaatcaccaatggtactggcaatgacatcaccacggctacaggatatgacactgctgatataattaagaacaataccaactttaggggcatgtacatatgtggttcgctccagcgtgcctggggtgataagaaaatttccaacactgcctttatcaccactgccaccctagatggtgacaacaacaacaagatcaatccctgtaataccatagaccagtcaaagatcgtcgtgtttcaagacaaccatgttggaaagaaagcgcaaacctcagacgatacattggccctgcttggttacactggcattggtgagcaggccatcgggtctgatagggaccgggttgtgcgcatcagcactctccctgaaactggtgctcgaggcggtaaccacccaattttctacaagaactccattaaattgggatatgtaattaggtctattgatgtctttaattcacaaatcttgcacacttccagacagttatcgctaaatcattacctactcccacctgattcttttgccgtctatagaataattgactcaaatggctcgtggtttgatattggaattgatagtgatgggttctcttttgttggtgtttctggctttggtaaattagaatttcccctttctgcctcctacatgggaatacaattggcaaagatccggcttgcctctaacattaggagtcccatgactaagtta

SEQ ID NO: 2 - protein, 671 aa, Feline calicivirus
MCSTCANVLKYYDWDPHFKLVINPNNFLSVGFCSNPLMCCYPELLPEFGTVWDCDRSPLEIYLESILGDDEWASTFDAVDPVVPPMHWGAAGKIFQPHPGVLMHHLIGKVAAGWDPDLPLIRLEADDGSITAPEQGTMVGGVIAEPSAQMSTAADMATGKSVDSEWEAFFSFHTSVNWSTSETQGKILFKQSLGPLLNPYLEHLAKLYVAWSGSIEVRFSISGSGVFGGKLAAIVVPPGVDPVQSTSMLQYPHVLFDARQVEPVIFCLPDLRSTLYHLMSDTDTTSLVIMVYNDLINPYANDANSSGCIVTVETKPGPDFKFHLLKPPGSMLTHGSIPSDLIPKTSSLWIGNRYWSDITDFVIRPFVFQANRHFDFNQETAGWSTPRFRPISVTITEQNGAKLGIGVATDYIVPGIPDGWPDTTIPGELIPAGDYAITNGTGNDITTATGYDTADIIKNNTNFRGMYICGSLQRAWGDKKISNTAFITTATLDGDNNNKINPCNTIDQSKIVVFQDNHVGKKAQTSDDTLALLGYTGIGEQAIGSDRDRVVRISTLPETGARGGNHPIFYKNSIKLGYVIRSIDVFNSQILHTSRQLSLNHYLLPPDSFAVYRIIDSNGSWFDIGIDSDGFSFVGVSGFGKLEFPLSASYMGIQLAKIRLASNIRSPMTKL

SEQ ID NO: 3 - DNA, 1641 bp, Feline calicivirus
  CDS (1)..(1641)
gcggatgacgggtcaatcacagcacccgagcaaggaacaatggttggcggcgtcatcgctgaacccagcgcccagatgtcaacagctgctgatatggccaccgggaaaagcgttgattctgagtgggaggcattcttctcctttcacaccagcgtcaattggagtacatctgaaacccaaggaaagattctcttcaaacaatccttaggccctttgctcaacccatatctagaacaccttgctaagctatatgttgcgtggtctgggtcgattgaggttaggttctctatctctggctctggtgtctttggtgggaagctcgcagctattgttgtacctcctggggttgatccagtgcagagtacttcgatgctacaatacccccatgttttgtttgatgctcgtcaggtggaaccagttatcttctgtcttcctgatctaagaagcaccctgtaccaccttatgtctgacactgacactacatccttggtcattatggtgtacaatgatctcatcaatccctatgccaatgatgccaactcttctgggtgtattgtcactgtcgagacaaaacctggccctgacttcaagtttcacctccttaagccacccggatctatgctaacccatggctctatcccttctgatttaattcccaaaacatcttcgctctggatcggtaaccgctactggtcagacataactgattttgtgattcggccgtttgtcttccaagcaaatcgtcattttgactttaatcaagagaccgcagggtggagcacaccacggtttcggcctatatctgttaccattactgaacagaacggagcaaaattgggcattggggtggcaacagattacatagtgcctggaatccctgatggctggcctgacaccacaattcctggggagttgataccagctggtgattacgcaatcaccaatggtactggcaatgacatcaccacggctacaggatatgacactgctgatataattaagaacaataccaactttaggggcatgtacatatgtggttcgctccagcgtgcctggggtgataagaaaatttccaacactgcctttatcaccactgccaccctagatggtgacaacaacaacaagatcaatccctgtaataccatagaccagtcaaagatcgtcgtgtttcaagacaaccatgttggaaagaaagcgcaaacctcagacgatacattggccctgcttggttacactggcattggtgagcaggccatcgggtctgatagggaccgggttgtgcgcatcagcactctccctgaaactggtgctcgaggcggtaaccacccaattttctacaagaactccattaaattgggatatgtaattaggtctattgatgtctttaattcacaaatcttgcacacttccagacagttatcgctaaatcattacctactcccacctgattcttttgccgtctatagaataattgactcaaatggctcgtggtttgatattggaattgatagtgatgggttctcttttgttggtgtttctggctttggtaaattagaatttcccctttctgcctcctacatgggaatacaattggcaaagatccggcttgcctctaacattaggagtcccatgactaagtta

SEQ ID NO: 4 - protein, 547 aa, Feline calicivirus
ADDGSITAPEQGTMVGGVIAEPSAQMSTAADMATGKSVDSEWEAFFSFHTSVNWSTSETQGKILFKQSLGPLLNPYLEHLAKLYVAWSGSIEVRFSISGSGVFGGKLAAIVVPPGVDPVQSTSMLQYPHVLFDARQVEPVIFCLPDLRSTLYHLMSDTDTTSLVIMVYNDLINPYANDANSSGCIVTVETKPGPDFKFHLLKPPGSMLTHGSIPSDLIPKTSSLWIGNRYWSDITDFVIRPFVFQANRHFDFNQETAGWSTPRFRPISVTITEQNGAKLGIGVATDYIVPGIPDGWPDTTIPGELIPAGDYAITNGTGNDITTATGYDTADIIKNNTNFRGMYICGSLQRAWGDKKISNTAFITTATLDGDNNNKINPCNTIDQSKIVVFQDNHVGKKAQTSDDTLALLGYTGIGEQAIGSDRDRVVRISTLPETGARGGNHPIFYKNSIKLGYVIRSIDVFNSQILHTSRQLSLNHYLLPPDSFAVYRIIDSNGSWFDIGIDSDGFSFVGVSGFGKLEFPLSASYMGIQLAKIRLASNIRSPMTKL

SEQ ID NO: 5 - DNA, 1752 bp, Feline parvovirus
  CDS (1)..(1752)
atgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacgggtgggtggaaatcacagcaaactcaagcagacttgtacatttaaatatgccagaaagtgaaaattataaaagagtagttgtaaataatatggataaaactgcagttaaaggaaacatggctttagatgatattcatgtacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatgtatatcatggtacagatccagatgatgttcaattttatactattgaaaattctgtgccagtacacttactaagaacaggtgatgaatttgctacaggaacatttttttttgattgtaaaccatgtagattaacacatacatggcaaacaaatagagcattgggcttaccaccatttttaaattctttgcctcaatctgaaggagctactaactttggtgatataggagttcaacaagataaaagacgtggtgtaactcaaatgggaaatacagactatattactgaagctactattatgagaccagctgaggttggttatagtgcaccatattattcttttgaagcgtctacacaagggccatttaaaacacctattgcagcaggacgggggggagcgcaaacagatgaaaatcaagcagcagatggtgatccaagatatgcatttggtagacaacatggtcaaaaaactactacaacaggagaaacacctgagagatttacatatatagcacatcaagatacaggaagatatccagaaggagattggattcaaaatattaactttaaccttcctgtaacaaatgataatgtattgctaccaacagatccaattgggggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacgaatgaatatgatcctgatgcatctgctaatatgtcaagaattgtaacttattcagatttttggtggaaaggtaaattagtatttaaagctaaactaagagcatctcatacttggaatccaattcaacaaatgagcattaatgtagataaccaatttaactatgtaccaaataatattggagctatgaaaattgtatatgaaaaatctcaactagcacctagaaaattatat

SEQ ID NO: 6 - protein, 584 aa, Feline parvovirus
MSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNVYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPNNIGAMKIVYEKSQLAPRKLY

SEQ ID NO: 7 - DNA, 729 bp, Feline parvovirus
  CDS (1)..(729)
ggtacagatccagatgatgttcaattttatactattgaaaattctgtgccagtacacttactaagaacaggtgatgaatttgctacaggaacatttttttttgattgtaaaccatgtagattaacacatacatggcaaacaaatagagcattgggcttaccaccatttttaaattctttgcctcaatctgaaggagctactaactttggtgatataggagttcaacaagataaaagacgtggtgtaactcaaatgggaaatacagactatattactgaagctactattatgagaccagctgaggttggttatagtgcaccatattattcttttgaagcgtctacacaagggccatttaaaacacctattgcagcaggacgggggggagcgcaaacagatgaaaatcaagcagcagatggtgatccaagatatgcatttggtagacaacatggtcaaaaaactactacaacaggagaaacacctgagagatttacatatatagcacatcaagatacaggaagatatccagaaggagattggattcaaaatattaactttaaccttcctgtaacaaatgataatgtattgctaccaacagatccaattgggggtaaaacaggaattaactatactaatatatttaatacttatggtcctttaactgcattaaataatgtaccaccagtttatccaaatggtcaaatttgggataaagaatttgatactgac

SEQ ID NO: 8 - protein, 243 aa, Feline parvovirus
GTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTD

SEQ ID NO: 9 - DNA, 1860 bp, Feline parvovirus
  CDS (1)..(1860)
atggcacctccggcaaagagagccaggagaggacttgtgcctccaggttataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctacagattggggggggaaaataggacattatttttttagagctaaaaaagcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcaagcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattataaaagagtagttgtaaataatatggataaaactgcagttaaaggaaacatggctttagatgacactcatgtacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatatatcatggtacagatccagatgatgttcaattttatactattgaaaattctgtgccagtacacttactaagaacaggtgatgaatttgctacaggaacatttttttttgattgtaaaccatgtagactaacacatacatggcaaacaaacagagcattgggcttaccaccatttctaaattctttgcctcaatctgaaggagctactaactttggtgatataggagttcaacaagataaaagacgtggtgtaactcaaatgggaaatacagactatattactgaagctactattatgagaccagctgaggttggttatagtgcaccatattattcttttgaagcgtctacacaagggccatttaaaatacctattgcagcaggacgggggggagcgcaaacagatgaaaatcaagcagcagatggtgatccaagatatgcatttggtagacaacatggtcaaaaaactactacaacaggagaaacacctgagagatttacatatatagcacatcaagatacaggaagatatccagcaggagattggattcaaaatattaactttaaccttcctgtaacaaatgataatgtattgctaccaacagatccaattggaggtaaaacaggaatcaactatactaatatatttaatacttatggtcctttaactgcattaaataatgtaccaccagtttatccaaatggtcaaatttgggataaagaatttgatactgac

SEQ ID NO: 10 - protein, 620 aa, Feline parvovirus
MAPPAKRARRGLVPPGYKYLGPGNSLDQGEPTNPSDAAAKEHDEAYAAYLRSGKNPYLYFSPADQRFIDQTKDATDWGGKIGHYFFRAKKAIAPVLTDTPDHPSTSRPTKPTKRSKPPPHIFINLAKKKKAGAGQVKRDNQAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDTHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKIPIAAGRGGAQTDENQAADGDPRYAFGRQHGQKTTTTGETPERFTYIAHQDTGRYPAGDWIQNINFNLPVTNDNVLLPTDPIGGKTGINYTNIFNTYGPLTALNNVPPVYPNGQIWDKEFDTD

SEQ ID NO: 11 - DNA, 1431 bp, Feline parvovirus
  CDS (1)..(1431)
atgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattataaaagagtagttgtaaataatatggataaaactgcagttaaaggaaacatggctttagatgacactcatgtacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatatatcatggtacagatccagatgatgttcaattttatactattgaaaattctgtgccagtacacttactaagaacaggtgatgaatttgctacaggaacatttttttttgattgtaaaccatgtagactaacacatacatggcaaacaaacagagcattgggcttaccaccatttctaaattctttgcctcaatctgaaggagctactaactttggtgatataggagttcaacaagataaaagacgtggtgtaactcaaatgggaaatacagactatattactgaagctactattatgagaccagctgaggttggttatagtgcaccatattattcttttgaagcgtctacacaagggccatttaaaatacctattgcagcaggacgggggggagcgcaaacagatgaaaatcaagcagcagatggtgatccaagatatgcatttggtagacaacatggtcaaaaaactactacaacaggagaaacacctgagagatttacatatatagcacatcaagatacaggaagatatccagcaggagattggattcaaaatattaactttaaccttcctgtaacaaatgataatgtattgctaccaacagatccaattggaggtaaaacaggaatcaactatactaatatatttaatacttatggtcctttaactgcattaaataatgtaccaccagtttatccaaatggtcaaatttgggataaagaatttgatactgac

SEQ ID NO: 12 - protein, 477 aa, Feline parvovirus
MSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDTHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKIPIAAGRGGAQTDENQAADGDPRYAFGRQHGQKTTTTGETPERFTYIAHQDTGRYPAGDWIQNINFNLPVTNDNVLLPTDPIGGKTGINYTNIFNTYGPLTALNNVPPVYPNGQIWDKEFDTD

SEQ ID NO: 13 - DNA, 2829 bp, Feline herpesvirus 1
  CDS (1)..(2829)
atgtccactcgtggcgatcttgggaagcggcgacgagggagtcgttggcagggacacagtggctattttcgacagagatgttttttcccttctctactcggtattgcagcgactggctccagacatggtaacggatcgtcgggattaaccagactagctagatatgtttcatttatctggatcgtactattcttagtcggtccccgtccagtagagggtcaatctggaagcacatcggaacaaccccggcggactgtagctacccctgaggtaggggtacaccaccaaaaccaactacagatcccaccgatatgtcgatatgaggaagctctccgtgcgtcccaaatagaggctaacggaccatcgactttttatatgtgtccaccaccttcaggatctactgtcgtgcgtttagagccaccacgggcctgtccagattataaactagggaaaaattttaccgagggtatagctgtaatatttaaagaaaatatagcgccatataaattcaaggcaaatatatactataaaaacattattatgacaacggtatggtctgggagttcctatgccgttacaaccaaccgatatacagacagggttcccgtgaaagttcaagagattacagatctcatagatagacggggtatgtgcctctcgaaagctgattacgttcgtaacaattatcaatttacggcctttgatcgagacgaggatcccagagaactgcctctgaaacctccaagttcaacactctccagagtccgtggatggcacaccaatgaaacatacacaaagatcgtgctgctggatttccaccactctgggacctctgtaaattgcatcgtagaggaagtggatgcaagatctgtatatccatatgactcatttgctatctccactggtgacgtgattcacatgtctccattctttgggctgagggatggagcccatgtagaacatactagttattcttcagacagatttcaacaaatcgagggatactatccaatagacttggataccgattacactggggcaccagtttctcgcaattttttggaaactccgcatgtgacagtggcctggaactggaccccaaagtctggtcgggtatgtaccttagccaaatggagggaaatagatgaaatgctaccgatgaatataggctcctatagatttacagccaagaccatatccgctactttcatctccaatacttcacaatttgaaatcaatcgtatccgtttgggggactgtgccaccaaggaggcagccgaagccatagaccggatttataagagtaaatatagtaaaactcatattcagactggaaccctggagacctacctagcccgtgggggatttctaatagctttccgtcccatgatcagcaacgaactagcaaagttatatatcaatgaattagcacgttccaatcgcacggtagtggatctcagtgcactcctcaatccatctggggaaacagtacaacgaactagaagatcggtcccatctaatcaacatcataggtcgcggcgcagcacaatagaggggggtatagaaaccgtgaacaatgcatcactcctcaagaccacctcatctgtggaattcgcaatgctacaatttgcctatgactacatacaagcccatgtaaatgaaatgttgagtcggatagccactgcctggtgtacacttcagaaccgcgaacatgtgctgtggacagagaccctaaaactcaatcccggtggggtggtctcgatggccctagaacgtcgtgtatccgcgcgcctacttggagatgccgtcgccgtaacacaatgtgttaacatttctagcggacatgtctatatccaaaattctatgcgggtgacgggttcatcaacgacatgttacagccgccctcttgtttccttccgtgccctcaatgactccgaatacatagaaggacaactaggggaaaacaatgaacttctcgtggaacgaaaactaattgagccttgcactgtcaataataagcggtattttaagtttggggcagattatgtatattttgaggattatgcgtatgtccgtaaagtcccgctatcggagatagaactgataagtgcgtatgtgattaaatctactctcctagaggatcgtgaatttctccactcaagttatacacgagctgagctggaagataccggcccttttgactacagcgagattcaacgccgcaaccaactccacgccttaaaattttatgatatagacagcatagtcagagtggataataatcttgtcatcatgcgtggtatggcaaatttttttcagggactcggggatgtgggggctggtttcggcaaggtggtcttaggggctgcgagtgcggtaatctcaacagtatcaggcgtatcatcatttctaaacaacccatttggagcattggccgtgggactgttaatattagctggcatcgtcgcagcattcctggcatatcgctatatatctagattacgtgcaaatccaatgaaagccttatatcctgtgacgactaggaatttgaaacagacggctaagagccccgcctcaacggctggtggggatagcgacccgggagtcgatgacttcgatgaggaaaagctaatgcaggcaagggagatgataaaatatatgtccctcgtatcggctatggagcaacaagaacataaggcgatgaaaaagaataagggcccagcgatcctaacgagtcatctcactaacatggccctccgtcgccgtggacctaaataccaacgcctcaataatcttgatagcggtgatgatactgaaacaaatcttgtc

SEQ ID NO: 14 - protein, 943 aa, Feline herpesvirus 1
MSTRGDLGKRRRGSRWQGHSGYFRQRCFFPSLLGIAATGSRHGNGSSGLTRLARYVSFIWIVLFLVGPRPVEGQSGSTSEQPRRTVATPEVGVHHQNQLQIPPICRYEEALRASQIEANGPSTFYMCPPPSGSTVVRLEPPRACPDYKLGKNFTEGIAVIFKENIAPYKFKANIYYKNIIMTTVWSGSSYAVTTNRYTDRVPVKVQEITDLIDRRGMCLSKADYVRNNYQFTAFDRDEDPRELPLKPPSSTLSRVRGWHTNETYTKIVLLDFHHSGTSVNCIVEEVDARSVYPYDSFAISTGDVIHMSPFFGLRDGAHVEHTSYSSDRFQQIEGYYPIDLDTDYTGAPVSRNFLETPHVTVAWNWTPKSGRVCTLAKWREIDEMLPMNIGSYRFTAKTISATFISNTSQFEINRIRLGDCATKEAAEAIDRIYKSKYSKTHIQTGTLETYLARGGFLIAFRPMISNELAKLYINELARSNRTVVDLSALLNPSGETVQRTRRSVPSNQHHRSRRSTIEGGIETVNNASLLKTTSSVEFAMLQFAYDYIQAHVNEMLSRIATAWCTLQNREHVLWTETLKLNPGGVVSMALERRVSARLLGDAVAVTQCVNISSGHVYIQNSMRVTGSSTTCYSRPLVSFRALNDSEYIEGQLGENNELLVERKLIEPCTVNNKRYFKFGADYVYFEDYAYVRKVPLSEIELISAYVIKSTLLEDREFLHSSYTRAELEDTGPFDYSEIQRRNQLHALKFYDIDSIVRVDNNLVIMRGMANFFQGLGDVGAGFGKVVLGAASAVISTVSGVSSFLNNPFGALAVGLLILAGIVAAFLAYRYISRLRANPMKALYPVTTRNLKQTAKSPASTAGGDSDPGVDDFDEEKLMQAREMIKYMSLVSAMEQQEHKAMKKNKGPAILTSHLTNMALRRRGPKYQRLNNLDSGDDTETNLV

SEQ ID NO: 15 - DNA, 750 bp, Feline herpesvirus 1
  CDS (1)..(750)
atgtccactcgtggcgatcttgggaagcggcgacgagggagtcgttggcagggacacagtggctattttcgacagagatgttttttcccttctctactcggtattgcagcgactggctccagacatggtaacggatcgtcgggattaaccagactagctagatatgtttcatttatctggatcgtactattcttagtcggtccccgtccagtagagggtcaatctggaagcacatcggaacaaccccggcggactgtagctacccctgaggtaggggtacaccaccaaaaccaactacagatcccaccgatatgtcgatatgaggaagctctccgtgcgtcccaaatagaggctaacggaccatcgactttttatatgtgtccaccaccttcaggatctactgtcgtgcgtttagagccaccacgggcctgtccagattataaactagggaaaaattttaccgagggtatagctgtaatatttaaagaaaatatagcgccatataaattcaaggcaaatatatactataaaaacattattatgacaacggtatggtctgggagttcctatgccgttacaaccaaccgatatacagacagggttcccgtgaaagttcaagagattacagatctcatagatagacggggtatgtgcctctcgaaagctgattacgttcgtaacaattatcaatttacggcctttgatcgagacgaggatcccagagaactgcctctgaaacctccaagttca

SEQ ID NO: 16 - protein, 250 aa, Feline herpesvirus 1
MSTRGDLGKRRRGSRWQGHSGYFRQRCFFPSLLGIAATGSRHGNGSSGLTRLARYVSFIWIVLFLVGPRPVEGQSGSTSEQPRRTVATPEVGVHHQNQLQIPPICRYEEALRASQIEANGPSTFYMCPPPSGSTVVRLEPPRACPDYKLGKNFTEGIAVIFKENIAPYKFKANIYYKNIIMTTVWSGSSYAVTTNRYTDRVPVKVQEITDLIDRRGMCLSKADYVRNNYQFTAFDRDEDPRELPLKPPSS

SEQ ID NO: 17 - DNA, 1602 bp, Feline herpesvirus 1
  CDS (1)..(1602)
atgagacgatataggatgggacgcggaatctaccttctctatatctgtctgttatatacatatctccagtttggtacttcgtcgacaaccgcggtcagtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattatattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatgtactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaatcaatgttggttagtgtcgttgctgtaatactaggagctgttctcatcacagtctttatctttattacggcattatgtttatattattctcatccccggcgatta

SEQ ID NO: 18 - protein, 534 aa, Feline herpesvirus 1
MRRYRMGRGIYLLYICLLYTYLQFGTSSTTAVSIENSDNSTAEMLSSTSMSATTPISQPTSPFTTPTRRSTNIATSSSTTQASQPTSTLTTLTRSSTTIATSPSTTQAATFIGSSTDSNTTLLKTTKKPKRKKNKNNGARFKLYCGYKGVIYRPYFSPLQLNCTLPTEPHITNPIDFEIWFKPRTRFGDFLGDKEDFVGNHTRTSILLFSSRNGSVNSMDLGDATLGILQSRIPDYTLYNIPIQHTEAMSLGIKSVESATSGVYTWRVYGGDVLNKTVLGQVNVSVVAYHPPSVNLTPRASLFNKTFEAVCAVANYFPPRSTKLTWYLDGKPIERQYISDTASVWIDGLITRSSVLAIPTTETDSEKPDIRCDLEWHESPVSYKRFTKSVAPDVYYPPTVSVTFADTRAICDVKCVPRDGISLMWKIGNYHLPKAMSADILITGPCIERPGLVNIQSMCDISETDGPVSYTCQTIGYPPILPGFYDTQVYDASPEIVSESMLVSVVAVILGAVLITVFIFITALCLYYSHPRRL

SEQ ID NO: 19 - DNA, 1401 bp, Feline herpesvirus 1
  CDS (1)..(1401)
agtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattatattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatgtactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaa

SEQ ID NO: 20 - protein, 467 aa, Feline herpesvirus 1
SIENSDNSTAEMLSSTSMSATTPISQPTSPFTTPTRRSTNIATSSSTTQASQPTSTLTTLTRSSTTIATSPSTTQAATFIGSSTDSNTTLLKTTKKPKRKKNKNNGARFKLYCGYKGVIYRPYFSPLQLNCTLPTEPHITNPIDFEIWFKPRTRFGDFLGDKEDFVGNHTRTSILLFSSRNGSVNSMDLGDATLGILQSRIPDYTLYNIPIQHTEAMSLGIKSVESATSGVYTWRVYGGDVLNKTVLGQVNVSVVAYHPPSVNLTPRASLFNKTFEAVCAVANYFPPRSTKLTWYLDGKPIERQYISDTASVWIDGLITRSSVLAIPTTETDSEKPDIRCDLEWHESPVSYKRFTKSVAPDVYYPPTVSVTFADTRAICDVKCVPRDGISLMWKIGNYHLPKAMSADILITGPCIERPGLVNIQSMCDISETDGPVSYTCQTIGYPPILPGFYDTQVYDASPEIVSE

SEQ ID NO: 21 - DNA, 1401 bp, Feline herpesvirus 1
  CDS (1)..(1401)
atgtccatcgaaaacagcgataatagtactgcggagatgttatcatctaccagcatgtccgctaccaccccgatatcccagccaacatctccattcactactccaactcgtcgctctacaaatatagctacatcctcttccaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattagattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacttctggtgtttatacatggcgtgtctatggtggagatggtctgaacaaaacagtgctgggtcaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtctcc

SEQ ID NO: 22 - protein, 467 aa, Feline herpesvirus 1
MSIENSDNSTAEMLSSTSMSATTPISQPTSPFTTPTRRSTNIATSSSTTQASQPTSTLTTLTRSSTTIATSPSTTQAATFIGSSTDSNTTLLKTTKKPKRKKNKNNGARFKLDCGYKGVIYRPYFSPLQLNCTLPTEPHITNPIDFEIWFKPRTRFGDFLGDKEDFVGNHTRTSILLFSSRNGSVNSMDLGDATLGILQSRIPDYTLYNIPIQHTEAMSLGIKSVESATSGVYTWRVYGGDGLNKTVLGQVNVSVVAYHPPSVNLTPRASLFNKTFEAVCAVANYFPPRSTKLTWYLDGKPIERQYISDTASVWIDGLITRSSVLAIPTTETDSEKPDIRCDLEWHESPVSYKRFTKSVAPDVYYPPTVSVTFADTRAICDVKCVPRDGISLMWKIGNYHLPKAMSADILITGPCIERPGLVNIQSMCDISETDGPVSYTCQTIGYPPILPGFYDTQVYDASPEIVS

SEQ ID NO: 23 - DNA, 1122 bp, Feline herpesvirus 1
  CDS (1)..(1122)
atgatgacacgtctacatttttggtggtgtggaatctttgcggtcctgaaatatctggtatgtacttcaagccttacgaccacgccaaaaacaactacggtttatgtgaagggatttaatatacctccactacgctacaattatactcaagccagaatcgtgccaaaaattccccaggcgatggatccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggtaccctataacccacaacctcaccataaagccgtactcttatattggtatcggactcatggccgagaacccccagtaagattccaagaggccattcgatatgatcgtcccgccataccgtctgggagtgaggattcgaaacggtccaacgactctagaggagaatcgagtggacccaattggatagacattgaaaattacactcctaaaaataatgtgcctattataatatctgacgatgacgttcctacagcccctcccaagggcatgaataatcagtcagtagtgatacccgcaatcgtactaagttgtcttataatagcactgattctaggagtgatatattatattttgagggtaaagaggtctcgatcaactgcatatcaacaacttcctataatacatacaactcaccatcct

SEQ ID NO: 24 - protein, 374 aa, Feline herpesvirus 1
MMTRLHFWWCGIFAVLKYLVCTSSLTTTPKTTTVYVKGFNIPPLRYNYTQARIVPKIPQAMDPKITAEVRYVTSMDSCGMVALISEPDIDATIRTIQLSQKKTYNATISWFKVTQGCEYPMFLMDMRLCDPKREFGICALRSPSYWLEPLTKYMFLTDDELGLIMMAPAQFNQGQYRRVITIDGSMFYTDFMVQLSPTPCWFAKPDRYEEILHEWCRNVKTIGLDGARDYHYYWVPYNPQPHHKAVLLYWYRTHGREPPVRFQEAIRYDRPAIPSGSEDSKRSNDSRGESSGPNWIDIENYTPKNNVPIIISDDDVPTAPPKGMNNQSVVIPAIVLSCLIIALILGVIYYILRVKRSRSTAYQQLPIIHTTHHP

SEQ ID NO: 25 - DNA, 900 bp, Feline herpesvirus 1
  CDS (1)..(900)
ccaaaaacaactacggtttatgtgaagggatttaatatacctccactacgctacaattatactcaagccagaatcgtgccaaaaattccccaggcgatggatccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggtaccctataacccacaacctcaccataaagccgtactcttatattggtatcggactcatggccgagaacccccagtaagattccaagaggccattcgatatgatcgtcccgccataccgtctgggagtgaggattcgaaacggtccaacgactctagaggagaatcgagtggacccaattggatagacattgaaaattacactcctaaaaataatgtgcctattataatatctgacgatgacgttcctacagcccctcccaagggcatgaataatcagtca

SEQ ID NO: 26 - protein, 300 aa, Feline herpesvirus 1
PKTTTVYVKGFNIPPLRYNYTQARIVPKIPQAMDPKITAEVRYVTSMDSCGMVALISEPDIDATIRTIQLSQKKTYNATISWFKVTQGCEYPMFLMDMRLCDPKREFGICALRSPSYWLEPLTKYMFLTDDELGLIMMAPAQFNQGQYRRVITIDGSMFYTDFMVQLSPTPCWFAKPDRYEEILHEWCRNVKTIGLDGARDYHYYWVPYNPQPHHKAVLLYWYRTHGREPPVRFQEAIRYDRPAIPSGSEDSKRSNDSRGESSGPNWIDIENYTPKNNVPIIISDDDVPTAPPKGMNNQS

SEQ ID NO: 27 - DNA, 759 bp, Feline leukemia virus
  CDS (1)..(759)
atgccgctgcgtgaaggtccgaacaaccgtccccagtattggccattctcagcttcagacctgtataactggaagtcgcataacccccctttctcccaagaccccgtggccctaactaacctaattgagtccattttagtgacgcatcaaccaacctgggacgactgccagcaactcttgcaggcactcctgacaggcgaagaaaggcaaagggtccttcttgaggcccgaaagcaggttccaggcgaggacggacggccaacccagctgcccaatgtcattgacgaagctttccccttgacccgtcccaactgggattttgctacgccggcaggtagggagcacctacgcctttatcgccagttgctgttagcgggtctccgcggggctgcaagacgccccactaatttggcacaggtaaagcaagttgtacaagggaaagaggaaacgccagcctcattcttagaaagattaaaagaggcttacagaatgtatactccctatgaccctgaggacccagggcaggctgctagtgttatcctgtcctttatctaccagtctagcccggacataagaaataagttacaaaggctagaaggcctacaggggttcacactgtctgatttgctaaaagaggcagaaaagatatacaacaaaagggagaccccagaggaaagggaagaaagattatggcagcggcaggaagaaagagataaaaagcgccataaggagatgactaaggtctgtgagaattctagc

SEQ ID NO: 28 - protein, 253 aa, Feline leukemia virus
MPLREGPNNRPQYWPFSASDLYNWKSHNPPFSQDPVALTNLIESILVTHQPTWDDCQQLLQALLTGEERQRVLLEARKQVPGEDGRPTQLPNVIDEAFPLTRPNWDFATPAGREHLRLYRQLLLAGLRGAARRPTNLAQVKQVVQGKEETPASFLERLKEAYRMYTPYDPEDPGQAASVILSFIYQSSPDIRNKLQRLEGLQGFTLSDLLKEAEKIYNKRETPEEREERLWQRQEERDKKRHKEMTKVCENSS

SEQ ID NO: 29 - DNA, 1830 bp, Feline leukemia virus
  CDS (1)..(1830)
atggccaatcctagtccaccccaaatgtataatgtaacttgggtaataaccaatgtacaaaccaacacccaagctaatgccacctctatgttaggaaccttaaccgatgtctaccctaccctacatgttgacttatgtgacctagtgggagacacctgggaacctatggtcctaagcccaaccgggtaccctccctcaaaatatggatgtaaaactacagatagaaaaaaacagcaacagacataccccttttacgtctgccccgggcatcgcccctcgctggggccaaagggaacacattgtggaggggcacaagatgggttttgtgccgcatggggatgtgaaaccaccggagaagcttggtggaagccctcctcctcatgggactatatcacagtaaaaagagggagtagtcagaacaataactgtgagggaaaatgcaaccccctgattttgcagttcacccagaaggggaaacaagcctcttgggacggacctaagatgtggggattgcggctataccgtacaggatatgaccctatcgccttattcacggtatcccggcgggtgtcaaccattacgccgcctcaggcaatgggaccagacctagtcttacctgatcaaaaacccccatcccgacaatctcaaacagggtccaaagtggcgacccagaggccccaaacgaatgaaagcgccccaaggtctgttgcccccaccaccgtgggtcccaaacggattgggaccggagataggttaataaatttagtacaaggggcatacctagccttaaatgccaccgaccccaacaaaactaaagactgttggctctgcctggtttctcgaccaccctattacgaagggattgcaatcttaggtaactacagcaaccaaacaaaccctcccccatcctgcctatctattccgccacacaagctgaccatatctaaagtatcagggcaaggactgtgcatagggactgttcctaagacccaccaggctttgtgcaataagacgcaccagggacatacaggggcggactatcgagccgccccgcggtatctagccgcccccaatggcacctattgggcctgtaacactggactcaccccatgcatttccatggcggtgctcaatttgacctctgatttttgtgtcttaatcgaattatggcccagagtgacttaccatcaacccgaatatgtgtacacacattttgccaaagctggcaggttccgaagagaaccaatatcactaactgttgccctcatgttgggaggactcactgtagggggcatagccgcgggggtcggaacagggactaaagccctccttgaaacagcccagttcagacaactacaaatggccatgcacacagacatccaggccctagaagagtcaattagtgccttagaaaagtccctgacctccctttctgaagtagtcttacaaaacagacggggcctagatattctattcctacaagagggagggctctgtgccgcattaaaagaagaatgttgcttctatgcggatcacaccggactcgtccgagacaatatggctaaattaagagaaagactaaaacagcggcaacaactgtttgactcccaacagggatggtttgaaggatggttcaacaggtccccctggtttacaaccctaatttcctccattatgggccccttactaatcctactcctaattctcctcttcggcccatacatccttaacagattagtacaattcgtaaaagacagaatatctgtggtacaagccttaattttaacccaacagtaccaacagataaagcaatacgatccggaccgacca

SEQ ID NO: 30 - protein, 610 aa, Feline leukemia virus
MANPSPPQMYNVTWVITNVQTNTQANATSMLGTLTDVYPTLHVDLCDLVGDTWEPMVLSPTGYPPSKYGCKTTDRKKQQQTYPFYVCPGHRPSLGPKGTHCGGAQDGFCAAWGCETTGEAWWKPSSSWDYITVKRGSSQNNNCEGKCNPLILQFTQKGKQASWDGPKMWGLRLYRTGYDPIALFTVSRRVSTITPPQAMGPDLVLPDQKPPSRQSQTGSKVATQRPQTNESAPRSVAPTTVGPKRIGTGDRLINLVQGAYLALNATDPNKTKDCWLCLVSRPPYYEGIAILGNYSNQTNPPPSCLSIPPHKLTISKVSGQGLCIGTVPKTHQALCNKTHQGHTGADYRAAPRYLAAPNGTYWACNTGLTPCISMAVLNLTSDFCVLIELWPRVTYHQPEYVYTHFAKAGRFRREPISLTVALMLGGLTVGGIAAGVGTGTKALLETAQFRQLQMAMHTDIQALEESISALEKSLTSLSEVVLQNRRGLDILFLQEGGLCAALKEECCFYADHTGLVRDNMAKLRERLKQRQQLFDSQQGWFEGWFNRSPWFTTLISSIMGPLLILLLILLFGPYILNRLVQFVKDRISVVQALILTQQYQQIKQYDPDRP

SEQ ID NO: 31 - DNA, 1833 bp, Feline leukemia virus
  CDS (1)..(1833)
atggagcacctacgcctttatcgccagttgctgttagcgggtctccgcggggctgcaagacaccccactaatttggcacaggttaagcaatttttacaagggaaagaagaaacgccagcctcattcttagaaagattaaaagaggcttaccgaatgtatactccctatgaccctgaggacccagggcaggctgctagtgttatcctgtcctttatctaccagtctagcccggacataagaaataagttacaaaggctagaaggcctacaggggttcacactgtctgatttgctaaaagaggcagaaaagatatacaacaaaagggagaccccagaggaaagggaagaaagattatggcagcggcaggaagaaagagataaaaagcgccataaggagatgactaaagttctggccacagtagttgctcagaatagagataaggatagagaggaaagtaaactgggagatcaaagaaaaatacctctggggaaagaccagtgtgcctattgcaaggaaaagggacattgggttcgcgattgccccaaccggccccggaagaaacccgccaactccactctcctcaacttagaagatatggccaatcctagtccaccccaaatgtataatgtaacttgggtaataaccaatgtacaaaccaacacccaagctaatgccacctctatgttaggaaccttaaccgatgtctaccctaccctacatgttgacttatgtgacctagtgggagacacctgggaacctatggtcctaagcccaaccgggtaccctccctcaaaatatggatgtaaaactacagatagaaaaaaacagcaacagacataccccttttacgtctgccccgggcatcgcccctcgctggggccaaagggaacacattgtggaggggcacaagatgggttttgtgccgcatggggatgtgaaaccaccggagaagcttggtggaagccctcctcctcatgggactatatcacagtaaaaagagggagtagtcagaacaataactgtgagggaaaatgcaaccccctgattttgcagttcacccagaaggggaaacaagcctcttgggacggacctaagatgtggggattgcggctataccgtacaggatatgaccctatcgccttattcacggtatcccggcgggtgtcaaccattacgccgcctcaggcaatgggaccagacctagtcttacctgatcaaaaacccccatcccgacaatctcaaacagggtccaaagtggcgacccagaggccccaaacgaatgaaagcgccccaaggtctgttgcccccaccaccgtgggtcccaaacggattgggaccggagataggttaataaatttagtacaaggggcatacctagccttaaatgccaccgaccccaacaaaactaaagactgttggctctgcctggtttctcgaccaccctattacgaagggattgcaatcttaggtaactacagcaaccaaacaaaccctcccccatcctgcctatctattccgccacacaagctgaccatatctaaagtatcagggcaaggactgtgcatagggactgttcctaagacccaccaggctttgtgcaataagacgcaccagggacatacaggggcggactatcgagccgccccgcggtatctagccgcccccaatggcacctattgggcctgtaacactggactcaccccatgcatttccatggcggtgctcaatttgacctctgatttttgtgtcttaatcgaattatggcccagagtgacttaccatcaacccgaatatgtgtacacacattttgccaaagctggcaggttccgaaga

SEQ ID NO: 32 - protein, 611 aa, Feline leukemia virus
MEHLRLYRQLLLAGLRGAARHPTNLAQVKQFLQGKEETPASFLERLKEAYRMYTPYDPEDPGQAASVILSFIYQSSPDIRNKLQRLEGLQGFTLSDLLKEAEKIYNKRETPEEREERLWQRQEERDKKRHKEMTKVLATVVAQNRDKDREESKLGDQRKIPLGKDQCAYCKEKGHWVRDCPNRPRKKPANSTLLNLEDMANPSPPQMYNVTWVITNVQTNTQANATSMLGTLTDVYPTLHVDLCDLVGDTWEPMVLSPTGYPPSKYGCKTTDRKKQQQTYPFYVCPGHRPSLGPKGTHCGGAQDGFCAAWGCETTGEAWWKPSSSWDYITVKRGSSQNNNCEGKCNPLILQFTQKGKQASWDGPKMWGLRLYRTGYDPIALFTVSRRVSTITPPQAMGPDLVLPDQKPPSRQSQTGSKVATQRPQTNESAPRSVAPTTVGPKRIGTGDRLINLVQGAYLALNATDPNKTKDCWLCLVSRPPYYEGIAILGNYSNQTNPPPSCLSIPPHKLTISKVSGQGLCIGTVPKTHQALCNKTHQGHTGADYRAAPRYLAAPNGTYWACNTGLTPCISMAVLNLTSDFCVLIELWPRVTYHQPEYVYTHFAKAGRFRR

SEQ ID NO: 33 - DNA, 1812 bp, canine distemper virus
  CDS (1)..(1812)
atgctcccctaccaagacaaggtgggtgccttctacaaggataatgcaagagccaattcaaccaagctgtccttagtgacagaaggacatgggggcaggagaccaccttatttgttgtttgtccttctcatcttattggttggtatcctggccttgcttgctatcactggagttcgatttcaccaagtatcaactagtaatatggaatttagcagattgctgaaagaggatatggagaaatcagaggccgtacatcaccaagtcatagatgtcttgacaccgctcttcaagattattggagatgagattgggttacggttgccacaaaagctaaacgagatcaaacaatttatccttcaaaagacaaatttcttcaatccgaacagagaattcgacttccgcgatctccactggtgcattaacccgcctagtacggtcaaggtgaattttactaattactgtgagtcaattgggatcagaaaagctattgcatcggcagcaaatcctatccttttatcagccctatctgggggcagaggtgacatattcccaccacacagatgcagtggagctactacttcagtaggcaaagttttccccctatcagtctcattatccatgtctttgatctcaagaacctcagaggtaatcaatatgctgaccgctatctcagacggcgtgtatggcaaaacttacttgctagtgcctgatgatatagaaagagagttcgacactcgagagattcgagtctttgaaatagggttcatcaaaaggtggctgaatgacatgccattactccaaacaaccaactatatggtactcccgaagaattccaaagccaaggtatgtactatagcagtgggtgagttgacactggcttccttgtgtgtagaagagagcactgtattattatatcatgacagcagtggttcacaagatggtattctagtagtgacactggggatattttgggcaacacctatggatcacattgaggaagtgatacctgtcgctcacccatcaatgaagaaaatacatataacaaaccaccgtggttttataaaagattcaattgcaacctggatggtgcctgccctggcctctgagaaacaagaagaacaaaaaggttgtctggagtcagcttgtcaaagaaaaacctaccccatgtgcaaccaagcgtcatgggaacccttcggaggaagacagttgccatcttatgggcggttgacattacctctagatgcaagtgttgaccttcaacttaacatatcgttcacatacggtccggttatactgaatggagatggtatggattattatgaaagcccacttttgaactccggatggcttaccattccccccaaagacggaacaatctctggattgataaacaaagcaggtagaggagaccagttcactgtactcccccatgtgttaacatttgcgcccagggaatcaagtggaaattgttatttacctattcaaacatctcaaattagagatagagatgtcctcattgagtccaatatagtggtgttgcctacacagagtattagatatgtcatagcaacgtatgacatatcacgaagtgatcatgctattgtttattatgtttatgacccaatccggacgatttcttatacgcacccatttagactaactaccaagggtagacctgatttcctaaggattgaatgttttgtgtgggatgacaatttgtggtgtcaccaattttacagattcgaggctgacatcgccaactctacaaccagtgttgagaatttagtccgtataagattctcatgtaaccgt

SEQ ID NO: 34 - protein, 604 aa, canine distemper virus
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 35 - DNA, 1986 bp, canine distemper virus
  CDS (1)..(1986)
atgcacaggggaatccccaaaagctccaaaacccaaacacatacccaacaagaccgccccccacaacccagcaccgaactcgaagagaccaggacctcccgagcacgacacagcacaacatcagctcagcgatccacgcactacgatcctcgaacatcggacagacccgtctcctacaccatgaacaggaccaggtcccgcaagcaaaccagccacagattgaagaacatcccagttcacggaaaccacgaggccaccatccagcacataccagagagtgtctcaaaaggagcgagatcccagatcgaaaggcggcaacccaatgcaatcaactcaggctctcattgcacctggttagtcctgtggtgcctcggaatggccagtctctttctttgttccaaggctcagatacattgggataatttgtcaactattgggattatcgggactgataatgtccattacaagatcatgactaggcccagtcaccagtacttggtcataaaattgatccctaatgcttcacttatagagaattgtaccaaagcagaattaggtgagtatgagaaattattgaattcagtcctcgaaccaatcaaccaagctttgactctaatgaccaagaatgtgaagcccctgcagtcattagggtcaggtaggagacaaaggcgttttgcaggagtggtacttgcaggtgtagctttaggagtggctacagctgcacaaatcactgcaggaatagctttacatcaatccaacctcaatgctcaagcaatccaatctcttagaaccagccttgaacagtctaacaaagctatagaagaaattagggaggctacccaagaaaccgtcattgccgttcagggagtccaggactacgtcaacaacgaactcgtccctgccatgcaacatatgtcatgtgaattagttgggcagagattagggttaagactgcttcggtattatactgagttgttgtcaatatttggcccgagtttacgtgaccctatttcagccgagatatcaattcaggcactgatttatgctcttggaggagaaattcataagatacttgagaagttgggatattctggaagtgatatgattgcaatcttggagagtcgggggataaaaacaaaaataactcatgttgatcttcccgggaaattcatcatcctaagtatctcatacccaactttatcagaagtcaagggggttatagtccacagactggaagcagtttcttacaacataggatcacaagagtggtacaccactgtcccgaggtatattgcaactaatggttacttaatatctaattttgatgagtcatcttgtgtattcgtctcagagtcagccatttgtagccagaactccctgtatcccatgagcccactcttacaacaatgtattaggggcgacacttcatcttgtgctcggaccttggtatctgggactatgggcaacaaatttattctgtcaaaaggtaatatcgtcgcaaattgtgcttctatactatgtaagtgttatagcacaagcacaattattaatcagagtcctgataagttgctgacattcattgcctccgatacctgcccactggttgaaatagatggtgctactatccaagttggaggcaggcaataccctgatatggtatacgaaggcaaagttgccttaggccctgctatatcacttgataggttagatgtaggtacaaacttagggaacgcccttaagaaactggatgatgctaaggtactgatagactcctctaaccagatccttgagacggttaggcgctcttcctttaattttggcagtctcctcagcgttcctatattaagttgtacagccctggctttgttgttgctgatttactgttgtaaaagacgctaccaacagacactcaagcagcatactaaggtcgatccggcatttaaacctgatctaactggaacttcgaaatcctatgtgagatcactc

SEQ ID NO: 36 - protein, 662 aa, canine distemper virus
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLKQHTKVDPAFKPDLTGTSKSYVRSL

(19) United States
(12) Patent Application Publication
(10) Pub. No.: US 2008/0286295 A1
(43) Pub. Date: Nov. 20, 2008

(54) USE OF RECOMBINANT ANTIGENS TO DETERMINE THE IMMUNE STATUS OF AN ANIMAL

(76) Inventors: Wayne A. Jensen, Wellington, CO (US); Michael R. Lappin, Fort Collins, CO (US); David K. Rosen, Portage, MI (US); Janet S. Andrews, Fort Collins, CO (US)

Correspondence Address: HESKA CORPORATION;LEGAL DEPARTMENT, 3760 ROCKY MOUNTAIN AVE, LOVELAND, CO, 80538, US

(21) Appl. No.: 12/128,862

(22) Filed: May 29, 2008

Related U.S. Application Data
(62) Division of application No. 10/670,695, filed on Sep. 25, 2003.
Application No. 10/670,695 is a continuation of application No. 09/521,738, filed on Mar. 9, 2000, now abandoned.

(51) Int. Cl. A61K 39/00; A61P 37/00

(52) U.S. Cl. 424/187.1; 424/184.1; 424/204.1; 424/213.1; 424/231.1; 424/224.1; 424/233.1; 424/186.1; 424/207.1

(57) ABSTRACT

The present invention includes a method to determine the immune status of an animal that includes the steps of (a) contacting a biological specimen of the animal with a recombinant infectious agent antigen that is specific for detecting an antibody selective for that infectious agent, under conditions suitable for formation of a complex between the recombinant antigen and the antibody and (b) detecting the presence or absence of the complex, wherein presence or absence of a complex is indicative of the immune status of the animal. Preferably such a method indicates whether the animal should be vaccinated. The present invention also includes an assay comprising (a) a recombinant infectious agent antigen that is specific for detecting an antibody selective for that infectious agent; and (b) a means to detect an antibody that selectively binds to the recombinant antigen. Also included in the present invention are recombinant antigens and nucleic acid molecules encoding such antigens as well as methods to produce and use such nucleic acid molecule and recombinant antigens.

### CROSS REFERENCE TO RELATED APPLICATIONS

This application is a Divisional of co-pending U.S. patent application Ser. No. 10/670,695, filed Sep. 25, 2003, entitled “USE OF RECOMBINANT ANTIGENS TO DETERMINE THE IMMUNE STATUS OF AN ANIMAL”; which is a Continuation of U.S. patent application Ser. No. 09/521,738, filed Mar. 9, 2000, entitled “USE OF RECOMBINANT ANTIGENS TO DETERMINE THE IMMUNE STATUS OF AN ANIMAL,” now abandoned.

### FIELD OF THE INVENTION

The present invention relates generally to materials and methods useful for the detection of antibodies in an animal. In particular, the invention relates to the use of recombinant antigens to determine the immune status of an animal in order to determine whether the animal has antibodies indicative of protection from infection by an infectious agent.

### BACKGROUND OF THE INVENTION

The need for vaccinations against pathogens has long been recognized in humans and other animals. The long term efficacy of vaccines, especially vaccines against viruses, has become a topic of interest more recently. One recent study, for example, showed that neutralizing antibody titers against feline parvovirus (FPV), feline herpesvirus (FHV), and feline calicivirus (FCV) remain in cats for at least three years following vaccination; see Scott, et al., 1997, Feline Practice 25, 12-19. The antibody titers do decline over time, however, and the exact time that any given cat remains protected against disease cannot be predicted without testing. Current guidelines for vaccination recommend that cats be revaccinated every three years; see, for example, Elston, et at, 1998, Feline Practice 26, 14-16; Elston, et al., 1998, J. Am. Vet. Med. Assoc. 212, 227-241. For dogs, the current recommendation is to revaccinate against canine parvovirus and canine distemper virus yearly.

Vaccinations, however, are not risk-free. Anaphylaxis, post-vaccine canine distemper encephalitis, polyarthritis, glomerulonephritis, immune-mediated hemolytic anemia, autoimmune nonregenerative anemia and immune-mediated thrombocytopenia are all reported adverse reactions to vaccinations; see, for example, McCaw, et al., 1998, J. Am. Vet. Med. Assoc. 213, 72-75. A small proportion of cats have also been reported to develop fibrosarcomas after multiple vaccine injections; see, for example, Hershey et al., 2000, J. Am. Vet. Med. Assoc. 216, 58-61. The risks associated with vaccination, coupled with recent research demonstrating that at least some cats may not require certain vaccinations for more than seven years and that at least some dogs may not require revaccination for more than two years, are indicative of the desirability of measuring antibody titers to determine the immune status of animals prior to vaccination; see, Scott, et al., 1999, Am. J. Vet. Res. 60, 652-58 1999; McCaw, et al., ibid.

The duration of immunity experiments performed by Scott, et al, 1999, ibid., and McCaw, et al, ibid, however, utilized virus neutralization (“VN”) tests to determine the amount of protective antibodies in test animals. Depending on the particular assay, VN tests typically require between three and four days to perform, and can require as long as six or seven days. Time is only one disadvantage of the VN test: the test also requires skilled laboratory personnel to perform, incurs significant cost, and involves the use of live virus, presenting a biohazard risk.

An enzyme-linked immunosorbent assay (ELISA) represents an alternative to VN tests. ELISAs usually require an overnight coating step, with the actual test being performed in less than one day. This test does require multiple steps and requires a relatively skilled technician for performance and analysis. Standard methods use whole virus or virus-infected cells as the antigen for the detection of protective antibodies, again posing a biohazard risk. See, for example, Hill, et al., 1995, Am. J. Vet. Res. 56, 1181-1187; Spencer, et al, 1991, J. Wildl. Dis. 27, 578-583; Fiscus, et al, 1985, Am. J. Vet. Res. 46, 859-63. Furthermore, whole virus preparations are contaminated with antigens from the cells used to grow the virus. The procedure for obtaining canine parvovirus (CPV) in Fiscus, et al., ibid., for example, is not sufficient to completely remove such cellular antigens from the preparation. When using a biological specimen such as blood or serum from a vaccinated animal as a test sample, cellular antigens in the virus preparation can react with antibodies previously produced by the animal in response to such cellular proteins being in the virus preparation with which the animal was previously vaccinated. The presence of such cellular antigens in an immunoassay frequently increases the level of the signal in the assay, thereby leading to false positive or ambiguous results.

Thus, the methods currently practiced to determine the immune status of an animal suffer from a number of disadvantages, which are multiplied with each antibody type that one wishes to detect. Accordingly, there remains a need for an improved assay for the detection of antibodies in a test sample that does not require the use of biohazardous material and does not utilize materials containing contaminants that lead to false positives. There also remains a need for an assay for the detection of antibodies to one or more infectious agents that can be performed in a relatively short time period, in a veterinarian's office, inexpensively, by unskilled personnel. There further remains a need for antigen reagents that not only are stable and economic to produce but also are consistent from batch to batch.

### SUMMARY OF THE INVENTION

The present invention relates generally to materials and methods useful for the detection of the immune status of an animal. In particular, the invention relates to recombinant antigens and their use as reagents to determine the presence of antibodies indicative of protection against disease in an animal.

One embodiment of the present invention is a method to determine the immune status of an animal. Such a method includes the steps of: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex, wherein presence or absence of a complex is indicative of the immune status of the animal. For example, presence of a complex indicates that the animal is not susceptible to (i.e., is protected from) infection by the infectious agent.

Another embodiment of the present invention is a method to determine whether to vaccinate an animal. Such a method includes the steps of: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex. Presence of such a complex indicates that the animal need not be vaccinated, whereas absence of such a complex indicates that the animal should be vaccinated.

Yet another embodiment of the present invention is an assay to determine the immune status of an animal. Such an assay includes (a) a recombinant infectious agent antigen that is specific for detecting an antibody selective for that infectious agent; and (b) a means to detect an antibody that selectively binds to the recombinant antigen.

The present invention also includes the following recombinant antigens: PFCVCP₆₇₁, PFCVCP₅₄₇, PFPVVP2₅₈₄, PFPVVP2C₂₄₃, PFPVpVP12₆₂₀, PFPVpVP2₄₇₇, PFHVgB₉₄₃, PFHVgB₂₅₀, PFHVgC₅₃₄, PFHVgC₄₆₇, PFHVgC₄₆₇₍ₒₚₜ₎, PFHVgD₃₇₄, PFHVgD₃₀₀, PFeLVp27₂₅₃, PFeLVp27₆₁₉, PFeLVp27-gp70₆₁₁, PCDVH⁶⁰⁴, and PCDVF₆₆₂. These recombinant antigens are represented, respectively by the following amino acid sequences: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36. Also included are nucleic acid molecules encoding such recombinant antigens as well as nucleic acid molecules fully complementary to such coding sequences. Also included are recombinant molecules and recombinant cells including such nucleic acid molecules as well as methods to produce such nucleic acid molecules, recombinant molecules, recombinant cells, and recombinant antigens.

### DETAILED DESCRIPTION OF THE INVENTION

The present invention includes a method to determine the immune status of an animal. As used herein, the phrase to determine the immune status of an animal refers to a method to detect antibodies in that animal that are selective for a given infectious agent. Presence of such antibodies indicates that the animal is protected from infection by the infectious agent. Such an animal need not be vaccinated as it is not susceptible to infection by the infectious agent. A method of the present invention to determine the immune status of an animal includes the steps of: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex, wherein presence or absence of a complex is indicative of the immune status of the animal. In one embodiment, such a method is used to determine whether to vaccinate an animal. The present invention also includes an assay to determine the immune status of an animal as well as recombinant antigens that can be used in such a method or assay. Also included are nucleic acid molecules encoding such recombinant antigens, recombinant molecules and recombinant cells as well as methods to produce and use such molecules and cells.

It was surprising to the inventors that recombinant antigens are essential to a method to accurately determine the immune status of an animal. Use of whole virus in such a method was found to be unacceptable due to the potential for false positives caused by cellular antigens co-purifying with the virus preparation. The problem with cellular antigens was compounded when virus was isolated in a large-scale preparation. Although attempts were made to overcome these problems, using, for example, ultracentrifugation or cesium chloride purification techniques to purify virus, unacceptable levels of cellular antigens remained. As described in more detail in the Examples, not only did reagents containing feline calicivirus (FCV), feline herpesvirus (FHV), or feline parvovirus (FPV) purified from Crandell feline kidney (CRFK) cells in which the respective virus had grown (i.e., FCV or FHV purified by ultracentrifigation or FPV through cesium chloride) yield positive results in an ELISA to detect antibodies in cats previously administered the respective virus, but so did the respective “control” reagents purified from uninfected CRFK cells in the same manner. Data obtained from the “control” reagents represented unacceptable false positive results, leading the inventors to pursue alternative routes to develop an immune status assay. The inventors subsequently found that a recombinantly produced viral antigen yields unexpectedly good results, with acceptable background levels, in the determination of the immune status of an animal by immunoassay.

As such, the present invention includes a method to determine the immune status of an animal that includes the following steps: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex, wherein presence or absence of a complex is indicative of the immune status of the animal. It is to be noted that the term “a” entity or “an” entity refers to one or more of that entity; for example, a recombinant antigen refers to one or more antigens or at least one antigen. As such, the terms “a” (or “an”), “one or more” and “at least one” can be used interchangeably herein. It is also to be noted that the terms “comprising”, “including”, and “having” can be used interchangeably.

As used herein, a recombinant infectious agent antigen is an antigen of an infectious agent that is produced using recombinant nucleic acid technology. Such an antigen, also referred to herein as a recombinant antigen of the present invention or simply as a recombinant antigen, can be identified in a straight-forward manner by its ability to specifically detect an antibody selective for that infectious agent. As used herein, an antibody selective for an infectious agent, also referred to herein as an anti-infectious agent antibody, is an antibody that selectively binds to that infectious agent in that it preferentially binds to that infectious agent as opposed to binding to a different, unrelated, infectious agent. It is to be noted that, in accordance with the present invention, such an antibody exists in a biological specimen of an animal because a given infectious agent, upon infecting the animal, induces an immune response that includes the production of such an antibody selective for that infectious agent. A recombinant antigen of the present invention is also able to specifically detect the presence of such an antibody in that the recombinant antigen is sufficiently similar to the corresponding antigen on the infectious agent to enable such detection. The specificity of such detection enables one to ascertain that an animal has antibodies to a given infectious agent rather than to an unrelated infectious agent. Binding of an antigen and antibody can be measured using a variety of methods known to those skilled in the art, such as, but not limited to, those methods disclosed elsewhere herein. Preferably, a recombinant antigen of the present invention has a binding affinity of from about 10⁸ liters per mole (⁻¹⁾ to about 10¹²⁻¹ for an anti-infectious agent antibody of the present invention.

A recombinant infectious agent antigen of the present invention can correspond exactly to the antigen as found on the infectious agent or the recombinant antigen can be a homolog of such a native antigen. Examples of homologs include proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homolog includes at least one epitope capable of forming an immunocomplex, also referred to herein as a complex, with an anti-infectious agent antibody. As used herein, the term epitope refers to the smallest portion of a protein or other antigen capable of selectively binding to the antigen binding site of an antibody. It is well accepted by those skilled in the art that the minimal size of a protein epitope is about four amino acids. In one embodiment, a recombinant antigen of the present invention is modified to produce a more soluble antigen. Methods to produce more soluble antigens by modifying either a nucleic acid sequence or the protein itself are well known to those skilled in the art. One example of such a method, not intended to be limiting, is protein iodoacetimidation.

A recombinant antigen homolog can be the result of natural allelic variation or natural mutation. Homologs of the present invention can also be produced using techniques known in the art including, but not limited to, direct modifications to the protein or modifications to the nucleic acid molecule encoding the protein using, for example, classic or recombinant nucleic acid molecule techniques to effect random or targeted mutagenesis.

It is to be appreciated that recombinant antigens of the present invention include, but are not limited to, full-length proteins, proteins that are encoded by allelic variants of a given nucleic acid sequence, hybrid proteins, fusion proteins, multivalent proteins, and proteins that are truncated homologs of, or are proteolytic products of, at least a portion of a protein. As used herein, the term hybrid protein refers to a single protein produced from at least two different proteins; i.e., having domains from at least two different proteins.

Due to the method by which it is produced, a recombinant antigen of the present invention is removed from its natural milieu. As such, a recombinant antigen is isolated or biologically pure. Such terms do not reflect the extent to which a recombinant antigen is purified. A preferred recombinant antigen is purified from the recombinant cell which expresses the protein. Examples of methods to produce recombinant antigens of the present invention are disclosed elsewhere herein.

A recombinant infectious agent antigen of the present invention is any recombinant antigen that corresponds to (e.g., is derived from) an infectious agent. Preferred is an infectious agent for which one desires to determine if an animal is susceptible to infection by that agent. Suitable infectious agents include, but are not limited to, viruses, bacteria, fungi, endoparasites and ectoparasites. As such, suitable recombinant infectious agent antigens include, but are not limited to, recombinant viral, bacterial, fungal, endoparasite and ectoparasite antigens. Examples of viral infectious agents include, but are not limited to, adenoviruses, caliciviruses, coronaviruses, distemper viruses, hepatitis viruses, herpesviruses, immunodeficiency viruses, infectious peritonitis viruses, leukemia viruses, oncogenic viruses, papilloma viruses, parainfluenza viruses, parvoviruses, rabies viruses, and reoviruses, as well as other cancer-causing or cancer-related viruses. Examples of bacterial infectious agents include, but are not limited to, Actinomyces, Bacillus, Bacteroides, Bartonella, Bordetella, Borrelia, Brucella, Campylobacter, Capnocytophaga, Clostridium, Corynebacterium, Coxiella, Dermatophilus, Ehrlichia, Enterococcus, Escherichia, Francisella, Fusobacterium, Haemobartonella, Helicobacter, Klebsiella, L-form bacteria, Leptospira, Listeria, Mycobacteria, Mycoplasma, Neorickettsia, Nocardia, Pasteurella, Peptococcus, Peptostreptococcus, Proteus, Pseudomonas, Rickettsia, Rochalimaea, Salmonella, Shigella, Staphylococcus, Streptococcus, and Yersinia. Examples of fungal infectious agents include, but are not limited to, Absidia, Acremonium, Alternaria, Aspergillus, Basidiobolus, Bipolaris, Blastomyces, Candida, Chlamydia, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Example of protozoan parasite infectious agents include, but are not limited to, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium, Pneumocystis, Sarcocystis, Schistosoma, Theileria, Toxoplasma, and Trypanosoma. Examples of helminth parasite infectious agents include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca, Spirometra, Stephanofilaria, Strongyloides, Strongylus, Thelazia, Toxascaris, Toxocara, Trichinella, Trichostrongylus, Trichuris. Uncinaria, and Wuchereria. Examples of ectoparasite infectious agents include, but are not limited to, fleas; ticks, including hard ticks and soft ticks; flies, such as midges, mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs.

Preferred recombinant antigens of the present invention include an adenovirus protein, a calicivirus protein, a coronavirus protein, a distemper virus protein, a herpesvirus protein, an immunodeficiency virus protein, an influenza virus protein, a leukemia virus protein, a parvovirus protein, a rabies virus protein, a Bartonella protein, an Ehrlichia protein, a Haemobartonella protein, a Leptospira protein, a Streptococcus protein, a protozoan myeloecephalitis protein, a Dirofilaria protein, and a Giardia protein. More preferred recombinant antigens include a feline calicivirus protein, a feline coronavirus protein, a feline heipesvirus protein, a feline leukemia virus protein, a feline parvovirus protein, a canine adenovirus protein, a canine coronavirus protein, a canine distemper virus protein, a canine parvovirus protein, a rabies virus protein, an equine herpesvirus I protein, an equine herpesvirus IV protein, an equine influenza virus protein, a Streptococcus equii protein, and an Ehrlichia protein. Even more preferred recombinant antigens of the present invention include a feline calicivirus capsid protein (a rFCVCP protein), a feline herpesvirus glycoprotein B (gB) protein (a rFHVgB protein), a feline herpesvirus glycoprotein C (gC) protein (a rFHVgC protein), a feline herpesvirus glycoprotein D (gD) protein (a rFHV gD protein), a feline parvovirus VP12 protein (a rFPVVP 12 protein), a feline parvovirus VP2 protein (a rFPVVP2 protein), a feline leukemia virus p27 protein (a rFeLVp27 protein), a feline leukemia virus glycoprotein70 protein (a rFeLVgp70 protein), a p27/gp70 fusion protein (a rFeLVp27-gp70 protein), a canine distemper virus fusion protein (a rCDVF protein), and a canine distemper virus hemagglutinin protein (a rCDVH protein). Even more preferred recombinant antigens of the present invention include PFCVCP₆₇₁, PFCVCP₅₄₇, PFPVVP2₅₈₄, PFPVVP2C₂₄₃, PFPVpVP12₆₂₀, PFPVpVP2₄₇₇, PFHVgB₉₄₃, PFHVgB₂₅₀, PFHVgC₅₃₄, PFHVgC₄₆₇, PFHVgC₄₆₇₍ₒₚₜ₎, PFHVgD₃₇₄, PFHVgD₃₀₀, PFeLVp27₂₅₃, PFeLVp27₆₁₉, PFeLVp27-gp70₆₁₁, PCDVH₆₀₄, and PCDVF₆₆₂, the characteristics and production of which are described in the Examples. Such recombinant proteins have the following respective amino acid sequences: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36.

Particularly preferred recombinant antigens of the present invention include proteins having at least one of the following amino acid sequences: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36. Also preferred are recombinant antigens that are fragments of any of such antigens having such cited amino acid sequences, the fragments being able to bind to antibodies selective for the corresponding infectious agent. Preferred recombinant antigens can be encoded by nucleic acid molecules that: (a) have at least one of the following nucleic acid sequences: SEQ ID NO:1, SEQ ID NO:3, SEQ ID NO:5, SEQ ID NO:7, SEQ ID NO:9, SEQ ID NO:1, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35; (b) are degenerates of the nucleic acid sequences of (a); (c) are allelic variants of the nucleic acid sequences of (a); or (d) are fragments of any of the nucleic acid molecules of (a), (b), or (c). The foregoing SEQ ID NOs represent nucleic acid and amino acid sequences deduced according to methods disclosed in the Examples. It should be noted that since nucleic acid sequencing technology is not entirely error-free, the foregoing SEQ ID NOs, at best, represent apparent nucleic acid and amino acid sequences of certain nucleic acid molecules and recombinant antigens, respectively, of the present invention. In addition, variation seen in the foregoing SEQ ID NOs can also be due, at least in part, to allelic variation, which can be caused by, among other factors, genetic drift.

Additional preferred recombinant antigens of the present invention share at least about 70%, preferably at least about 75%, more preferably at least about 80%, more preferably at least about 85%, more preferably at least about 90%, more preferably at least about 95%, and more preferably about 100% identity at the amino acid level with a protein having at least one of the following amino acid sequences: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36. Also preferred are fragments of such antigens, and particularly fragments that are at least about 5, 10, 15, 20, 25, 30, 40, 50, 60, 70, 80, 90, 100, 150, 200, 250, 300, 350, 400, 450, 500, 550, 600, 650, 700, 750, 800, 850, or 900 amino acids in length.

The present invention also includes a recombinant antigen nucleic acid molecule. Recombinant antigen nucleic acid molecules of the present invention include any recombinant nucleic acid molecule that encodes a recombinant antigen of the present invention as well as a nucleic acid molecule fully complementary to any such coding sequence. A nucleic acid molecule of the present invention can be single-stranded or double-stranded. In accordance with the present invention, an isolated nucleic acid molecule is a nucleic acid molecule that has been removed from its natural milieu, i.e., that has been subjected to human manipulation, and can include DNA, RNA, or derivatives of either DNA or RNA. It is to be noted that the term isolated does not reflect the extent to which the nucleic acid molecule has been purified. A recombinant antigen nucleic acid molecule of the present invention can be isolated from its natural source or produced using recombinant DNA technology, e.g., polymerase chain reaction (PCR) amplification or cloning, or chemical synthesis. Although the phrase, nucleic acid molecule, primarily refers to the physical nucleic acid molecule and the phrase, nucleic acid sequence, primarily refers to the sequence of nucleotides on the nucleic acid molecule, the two phrases can be used interchangeably.

A nucleic acid molecule of the present invention can be a natural isolate or a homolog thereof. Nucleic acid molecule homologs include natural allelic variants and nucleic acid molecules modified by one or more nucleotide insertions, deletions, substitutions, and/or inversions in a manner such that the modification(s) do not substantially interfere with the nucleic acid molecule's ability to encode a recombinant antigen of the present invention. A nucleic acid molecule homolog of the present invention can be produced using a number of methods known to those skilled in the art; see, for example, Sambrook et al., 1989, Molecular Cloning: A Laboratory Manual, Cold Spring Harbor Labs Press; Sambrook et al., ibid., is incorporated by reference herein in its entirety. For example, nucleic acid molecules can be modified using a variety of techniques including, but not limited to, classic mutagenesis and recombinant DNA techniques such as site-directed mutagenesis, chemical treatment, restriction enzyme cleavage, ligation of nucleic acid fragments, PCR amplification, synthesis of oligonucleotide mixtures and ligation of mixture groups to build a mixture of nucleic acid molecules, and combinations thereof. Nucleic acid molecule homologs can be selected by hybridization or by screening for the function of a protein encoded by the nucleic acid molecule, e.g., ability to detect antibodies selective for the corresponding infectious agent.

Suitable and preferred nucleic acid molecules of the present invention encode suitable and preferred recombinant antigens as disclosed herein. Particularly preferred nucleic acid molecules of the present invention include the following nucleic acid sequences: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35; as well as nucleic acid molecules having nucleic acid sequences fully complementary to such sequences. Particularly preferred double-stranded nucleic acid molecules include nFCVCP₂₀₁₃, nFCVCP₁₆₄₁, nFPVVP2₁₇₅₂, nFPVVP2C₇₂₉, nFPVpVP12₁₈₆₀, nFPVpVP2₁₄₃₁, nFHVgB₂₈₂₉, nFHVgB₇₅₀, nFHVgC₁₆₀₂, nFHVgC₁₄₀₁, nFHVgC₁₄₀₁₍ₒₚₜ₎, nFHVgD₁₁₂₂, nFHVgD₉₀₀, nFeLVp27₇₅₉, nFeLVp27₁₈₅₇nFeLVp27-gp70₁₈₃₃, nCDVH₁₈₁₂, and nCDVF₁₉₈₆. Also preferred are nucleic acid molecules having degenerate sequences to any of the afore-mentioned nucleic acid molecules having cited nucleic acid sequences and nucleic acid molecules that are allelic variants thereof as well as fragments of any of the above-mentioned nucleic acid molecules. As used herein a nucleic acid molecule having a sequence that is degenerate as compared to a cited nucleic acid sequence is a nucleic acid molecule that encodes the same protein as the nucleic acid molecule having the cited sequence, but has a different nucleic acid sequence due to the degeneracy of the genetic code. As used herein, an allelic variant of a nucleic acid molecule having a cited nucleic acid sequence is a nucleic acid molecule that is a gene occurring at essentially the same locus (or loci) in the genome as the gene including the particular SEQ ID NO's cited herein, but which, due to natural variations caused by, for example, mutation or recombination, has a similar but not identical sequence. Also included in the term allelic variant are allelic variants of cDNAs derived from such genes. Because natural selection typically selects against alterations that affect function, allelic variants usually encode proteins having similar activity to that of the protein encoded by the gene to which they are being compared. Allelic variants of nucleic acid molecules can also comprise alterations in the 5′ or 3′ untranslated regions of the gene (e.g., in regulatory control regions), or can involve alternative splicing of a nascent transcript, thereby bringing alternative exons into juxtaposition. Allelic variants are well known to those skilled in the art and would be expected to be found within a given infectious agent.

Additional preferred recombinant antigen nucleic acid molecules of the present invention share at least about 70%, preferably at least about 75%, more preferably at least about 80%, more preferably at least about 85%, more preferably at least about 90%, more preferably at least about 95%, and more preferably about 100% identity at the nucleic acid level with a nucleic acid molecule having at least one of the following nucleic acid sequences: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35. Also preferred are fragments of such nucleic acid molecules, an particularly fragments that are at least about 15, 20, 25, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, or 2800 nucleotides in length.

The minimal size of a recombinant antigen of the present invention is a size sufficient to be encoded by a nucleic acid molecule capable of forming a stable hybrid (i.e., hybridize under stringent hybridization conditions) with the complementary sequence of a nucleic acid molecule encoding the corresponding protein. The size of a nucleic acid molecule encoding such a protein is dependent on the nucleic acid composition and the percent homology between the nucleic acid molecule and the complementary nucleic acid sequence. It can easily be understood that the extent of homology required to form a stable hybrid under stringent conditions can vary depending on whether the homologous sequences are interspersed throughout a given nucleic acid molecule or are clustered (i.e., localized) in distinct regions on a given nucleic acid molecule.

The minimal size of a nucleic acid molecule capable of forming a stable hybrid with a nucleic acid molecule encoding a recombinant antigen is typically at least about 12 to about 15 nucleotides in length if the nucleic acid molecule is GC-rich and at least about 15 to about 17 nucleotides in length if it is AT-rich. The minimal size of a nucleic acid molecule used to encode a recombinant antigen homolog of the present invention is from about 12 to about 18 nucleotides in length. Thus, the minimal size of a recombinant antigen homolog of the present invention is from about 4 to about 6 amino acids in length. There is no limit, other than a practical limit, on the maximal size of a nucleic acid molecule encoding a recombinant antigen of the present invention because a nucleic acid molecule of the present invention can include a portion of a full-length coding region, a full-length coding region, or multiple coding regions (either partial or full-length). The preferred size of a protein encoded by a nucleic acid molecule of the present invention depends on whether a full-length, fusion, multivalent, or functional portion of such a protein is desired.

Stringent hybridization conditions are determined based on defined physical properties of the target nucleic acid molecule to which a nucleic acid molecule is being hybridized, and can be defined mathematically. Stringent hybridization conditions are those experimental parameters that allow an individual skilled in the art to identify significant similarities between heterologous nucleic acid molecules, i.e., those conditions that allow the identification of nucleic acid molecules that are at least about 70% identical, or that share less than about 30% mismatch. These conditions are well known to those skilled in the art. See, for example, Sambrook, et al., 1989, ibid., and Meinkoth, et al., 1984, Anal. Biochem. 138, 267-284; Meinkoth, et al., is incorporated by reference herein in its entirety.

Furthermore, it is known in the art that there are commercially available computer programs for determining the degree of similarity between two nucleic acid sequences or amino acid sequences. These computer programs include various known methods to determine the percentage identity and the number and length of gaps between nucleic acid molecules and proteins. It is further known that the various available sequence analysis programs produce substantially similar results when the two compared molecules encode amino acid sequences that have greater than 30% amino acid identity. See Johnson et al., 1993J. Mol. Biol. 233, 716-738, 1993, and Feng et al., 1985, J. Mol. Evol. 21, 112-125, 1985, each of which is incorporated by reference herein in its entirety. Preferred methods to determine the percent identity among amino acid sequences and also among nucleic acid sequences include analysis using one or more of the commercially available computer programs designed to compare and analyze nucleic acid or amino acid sequences. These computer programs include, but are in no way limited to, GCG™ (available from Genetics Computer Group, Madison, Wis.), DNASIS® (available from Hitachi Software, San Bruno, Calif.) and MacVector (available from the Eastman Kodak Company, New Haven, Conn.). A particularly preferred method to determine the percent identity among amino acid sequences and also among nucleic acid sequences is to perform the analysis using the DNASIS® computer program, using default parameters.

The present invention also includes mimetopes of recombinant antigens of the present invention. In accordance with the present invention, a “mimetope” refers to any compound that is able to mimic the ability of a recombinant antigen of the present invention to bind to an antibody. A mimetope can be a peptide that has been modified to decrease its susceptibility to degradation but that still retains antibody-binding activity. Other examples of mimetopes include, but are not limited to, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of synthetic compounds for compounds capable of binding to anti-infectious agent antibodies. A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. The predicted mimetope structures can then be produced by, for example, chemical synthesis, recombinant DNA technology, or by isolation from a natural source.

One embodiment of the present invention includes a recombinant vector that includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulating of antigen nucleic acid molecules of the present invention.

One type of recombinant vector, referred to herein as a recombinant molecule, comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, insect and mammalian cells, and more preferably in bacteria.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, insect or mammalian cells. More preferred transcription control sequences include those that function in bacteria, such as, but not limited to, tac, lac, trp, trc, oxy-pro, omp/lpp, rrnB, bacteriophage lambda (such as lambda p_{L} and lambda p_{R} and fusions that include such promoters), bacteriophage T7, T7lac, bacteriophage T3, bacteriophage SP6, bacteriophage SP01, and antibiotic resistance gene transcription control sequences.

Suitable and preferred nucleic acid molecules to include in recombinant vectors of the present invention are as disclosed herein. Preferred nucleic acid molecules to include in recombinant vectors, and particularly in recombinant molecules, include nFCVCP₂₀₁₃, nFCVCP₁₆₄₁, nFPVVP2₁₇₅₂, nFPVVP2C₇₂₉, nFPVpVP12₁₈₆₀, nFPVpVP2₁₄₃₁, nFHVgB₂₈₂₉, nFHVgB₇₅₀, nFHVgC₁₆₀₂, nFHVgC₁₄₀₁, nFHVgC₁₄₀₁₍ₒₚₜ₎, nFHVgD₁₁₂₂, nFHVgD₉₀₀, nFeLVp27₇₅₉, nFeLVp27₁₈₅₇, nFeLVp27-gp70₁₈₃₃, nCDVH₁₈₁₂, and nCDVF₁₉₈₆. Particularly preferred recombinant molecules of the present invention include pλP_{R}His-nFCVCP₂₀₁₃, pλP_{R}-nFCVCP₁₆₄₁, pλP_{R}His-nFPVVP2₁₇₅₂, pλP_{R}His-nFPVVP2C₇₂₉, pλP_{R}-nFPVVP2C₇₂₉, pλP_{R}His-nFPVpVP12₁₈₆₀, pλP_{R}His-nFPVpVP2₁₄₃₁, pλP_{R}-nFPVpVP2₁₄₃₁, pλP_{R}His-nFHVgB₂₈₂₉, pλP_{R}His-nFHVgB₇₅₀, pλP_{R}His-nFHVgC₁₆₀₂, pλP_{R}His-nFHVgC₁₄₀₁, pλP_{R}-nFHVgC₁₄₀₁₍ₒₚₜ₎, pλP_{R}His-nFHVgD₁₁₂₂, pλP_{R}His-nFHVgD₉₀₀, pλP_{R}-nFeLVp27₇₅₉, pλP_{R}His-nFeLVp27₁₈₅₇, pλP_{R}-nFeLVp27-gp70₁₈₃₃, pλP_{R}His-nCDVH₁₈₁₂, and pλP_{R}His-nCDVF₁₉₈₆, the production of which are described in the Examples section.

Recombinant molecules of the present invention may also (a) contain secretory signals (i.e., signal segment nucleic acid sequences) to enable an expressed antigen of the present invention to be secreted from the cell that produces the protein and/or (b) contain fusion sequences which lead to the expression of nucleic acid molecules of the present invention as fusion proteins. Examples of suitable signal segments include any signal segment capable of directing the secretion of a protein of the present invention. Suitable fusion segments for use with the present invention include, but are not limited to, segments that can: enhance a protein's stability, enhance attachment of a protein to a substrate, and/or assist purification of a isolated antigen of the present invention (e.g., by affinity chromatography). A suitable fusion segment can be a domain of any size that has the desired function (e.g., imparts increased stability, enhances attachment to a substrate, and/or simplifies purification of a protein). Fusion segments can be joined to amino and/or carboxyl termini of the of the protein and can be susceptible to cleavage in order to enable straight-forward recovery of a isolated antigen of the present invention. Fusion proteins are preferably produced by culturing a recombinant cell transformed with a fusion nucleic acid molecule that encodes a protein including the fusion segment attached to either the carboxyl and/or amino terminal end of a domain. Preferred fusion segments include a metal binding domain (e.g., a poly-histidine segment); an immunoglobulin binding domain (e.g., Protein A; Protein G; T cell; B cell; Fc receptor or complement protein antibody-binding domains); a sugar binding domain (e.g., a maltose binding domain); and/or a “tag” domain (e.g., at least a portion of β-galactosidase, a strep tag peptide, other domains that can be purified using compounds that bind to the domain, such as monoclonal antibodies). A more preferred fusion segment is a metal binding domain. Examples of particularly preferred fusion proteins of the present invention include PHis-PFCVCP₆₇₁, PHis-PFCVCP₅₄₇, PHis-PFPVVP2₅₈₄, PHis-PFPVVP2C₂₄₃, PHis-PFPVpVP12₆₂₀, PHis-PFPVpVP2₄₇₇, PHis-PFHVgB₉₄₃, PHis-PFHVgB₂₅₀, PHis-PFHVgC₅₃₄, PHis-PFHVgC₄₆₇, PHis-PFHVgC₄₆₇₍ₒₚₜ₎, PHis-PFHVgD₃₇₄, PHis-PFHVgD₃₀₀, PHis-PFeLVp27₂₅₃, PHis-PFeLVp27₆₁₉, PHis-PFeLVp27-gp70₆₁₁, PHis-PCDVH₆₀₄, and PHis-PCDVF₆₆₂; methods to produce such fusion proteins are disclosed in the Examples. The present invention also includes post-translational modification of a recombinant antigen to introduce a ligand. Examples of ligands include biotin, biotin-like compounds, avidin, avidin-like compounds, metal binding compounds, sugar binding compounds, immunoglobulin binding domains, and other tag domains.

Another embodiment of the present invention includes a recombinant cell comprising a host cell transformed with one or more nucleic acid molecules or recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. Transformed nucleic acid molecules of the present invention can remain extrachromosomal or can integrate into one or more sites within a chromosome of the transformed (i.e., recombinant) cell in such a manner that their ability to be expressed is retained. Suitable nucleic acid molecules with which to transform a cell include any nucleic acid molecules disclosed herein that encode a recombinant antigen. Particularly preferred nucleic acid molecules with which to transform a cell include nFCVCP₂₀₁₃, nFCVCP₁₆₄₁, nFPVVP2₁₇₅₂, nFPVVP2C₇₂₉, nFPVpVP12₁₈₆₀, nFPVpVP2₁₄₃₁, nFHVgB₂₈₂₉, nFHVgB₇₅₀, nFHVgC₁₆₀₂, nFHVgC₁₄₀₁, nFHVgC₁₄₀₁₍ₒₚₜ₎, nFHVgD₁₁₂₂, nFHVgD₉₀₀, nFeLVp27₇₅₉, nFeLVp27₁₈₅₇, nFeLVp27-gp70₁₈₃₃, nCDVH₁₈₁₂, and nCDVF₁₉₈₆.

Suitable host cells to transform include any cell that can be transformed with a nucleic acid molecule of the present invention. Host cells can be either untransformed cells or cells that are already transformed with at least one nucleic acid molecule. Host cells of the present invention can be any cell capable of producing at least one protein of the present invention, and include bacterial, fungal (including yeast), parasite (including helminth, protozoa and ectoparasite), other insect, other animal and plant cells. Preferred host cells include bacterial, mycobacterial, yeast, insect and mammalian cells. More preferred host cells include Salmonella, Escherichia, Bacillus, Listeria, Saccharomyces, Pichia, Spodoptera, Mycobacteria, and Trichoplusia cells. Particularly preferred host cells are Escherichia coli.

A recombinant cell is preferably produced by transforming a host cell with a recombinant molecule encoding a recombinant antigen of the present invention operatively linked to an expression vector containing a transcription control sequence. Particularly preferred recombinant molecules include pλP_{R}His-nFCVCP₂₀₁₃, pλP_{R}-nFCVCP₁₆₄₁, pλP_{R}His-nFPVVP2₁₇₅₂, pλP_{R}His-nFPVVP2C₇₂₉, pλP_{R}-nFPVVP2C₇₂₉, pλP_{R}His-nFPVpVP12₁₈₆₀, pλP_{R}His-nFPVpVP2₁₄₃₁, pλP_{R}-nFPVpVP2₁₄₃₁, pλP_{R}His-nFHVgB₂₈₂₉, pλP_{R}His-nFHVgB₇₅₀, pλP_{R}His-nFHVgC₁₆₀₂, pλP_{R}His-nFHVgC1₄₀1, pλP_{R}-nFHVgC₁₄₀₁₍ₒₚₜ₎, pλP_{R}HiS-nFHVgD₁₁₂₂, pλP_{R}HiS-nFHVgD₉₀₀, pλP_{R}-nFeLVp27₇₅₉, pλP_{R}His-nFeLVp27₁₈₅₇, pλP_{R}-nFeLVp27-gp70₁₈₃₃, pλP_{R}His-nCDVH₁₈₁₂, and pλP_{R}His-nCDVF₁₉₈₆. Particularly preferred recombinant cells include E. coli:pλP_{R}His-nFCVCP₂₀₁₃, E. coli:pλP_{R}-nFCVCP₁₆₄₁, E. coli:pλP_{R}His-nFPVVP2₁₇₅₂, E. coli:pλP_{R}His-nFPVVP2C₇₂₉, E. coli:pλP_{R}-nFPVVP2C₇₂₉, E. coli:pλP_{R}His-nFPVpVP12₁₈₆₀, E. coli:pλP_{R}His-nFPVpVP2₁₄₃₁, E. coli:pλP_{R}— nFPVpVP2₁₄₃₁, E. coli:pλP_{R}His-nFHVgB_{2829,} E. coli:pλP_{R}His-nFHVgB₇₅₀, E. coli:pλP_{R}His-nFHVgC₁₆₀₂, E. coli:pλP_{R}His-nFHVgC₁₄₀₁, E. coli:pλP_{R}-nFHVgC₁₄₀₁₍ₒₚₜ₎, E. coli:pλP_{R}His-nFHVgD₁₁₂₂, E. coli:pλP_{R}His-nFHVgD₉₀₀, E. coli:pλP_{R}-nFeLVp27₇₅₉, E. coli:pλP_{R}His-nFeLVp27₁₈₅₇, E. coli:pλP_{R}-nFeLVp27-gp70₁₈₃₃, E. coli:pλP_{R}His-nCDVH₁₈₁₂, and E. coli:pλP_{R}His-nCDVF₁₉₈₆. Details regarding the production of these recombinant cells are disclosed herein.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant antigen of the present invention may be improved by fragmenting, modifying, or derivatizing a nucleic acid molecule encoding such an antigen.

Recombinant antigens of the present inventions can be produced in a variety of ways known to those skilled in the art. In one embodiment, a recombinant antigen of the present invention is produced by culturing a cell capable of expressing the antigen under conditions effective to produce the antigen, and recovering the antigen. A preferred cell to culture is a recombinant cell of the present invention. Effective culture conditions include, but are not limited to, effective media, bioreactor, temperature, pH and oxygen conditions that permit protein production. An effective, medium refers to any medium in which a cell is cultured to produce a recombinant antigen of the present invention. Such medium typically comprises an aqueous medium having assimilable carbon, nitrogen and phosphate sources, and appropriate salts, minerals, metals and other nutrients, such as vitamins, Recombinant cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. Examples of suitable conditions are included in the Examples section.

Depending on the vector and host system used for production, the expressed recombinant antigens may either remain within the recombinant cell; be secreted into the fermentation medium; be secreted into a space between two cellular membranes, such as the periplasmic space in E. coli; or be retained on the outer surface of a cell or viral membrane.

The phrase “recovering the antigen”, as well as similar phrases, refers to collecting the whole fermentation medium containing the recombinant product and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, Concanavalin A chromatography, chromatofocusing and differential solubilization. Recombinant antigens of the present invention are preferably retrieved in “substantially pure” form. As used herein, “substantially pure” refers to a purity that allows for the effective use of the protein as a detection reagent. Preferably, such a recombinant antigen reagent does not cause false positive reactions. In a preferred embodiment, recombinant antigens of the present invention are at least about 60% pure, preferably at least about 65% pure, more preferably at least about 70% pure, more preferably at least about 75% pure, more preferably at least about 80% pure, more preferably at least about 85% pure, more preferably at least about 90% pure, and more preferably at least about 95% pure. In one embodiment, a recombinant antigen of the present invention is at least about 98% to 100% pure.

One embodiment of the present invention is a method to determine the immune status of an animal to a desired infectious agent by detecting antibodies in that animal that selectively bind to that infectious agent. The method includes the steps of: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex, wherein presence or absence of a complex is indicative of the immune status of the animal. Presence of a complex indicates that an animal is protected from, or is not susceptible to, infection by that infectious agent, and as such, that animal need not be vaccinated. Absence of a complex suggests that an animal may not be protected from, or may be susceptible to, infection by that infectious agent, and as such, it is desirable to vaccinate that animal.

Antibodies to be detected can be maternal antibodies transferred to the offspring or can be generated (i.e., produced) in response to a natural infection by an infectious agent or vaccination. Vaccination can be accomplished in a variety of ways known to those skilled in the art including, but not limited to, administering the infectious agent itself or any immunogenic form thereof, such as, but not limited to, a modified live infectious agent, an inactivated, disrupted, fractionated or attenuated infectious agent, a native or recombinant antigen, or a nucleic acid molecule that invokes an immune response against the infectious agent. Antibodies to be detected can be of any class, i.e., immunoglobulin A (IgA), immunoglobulin D (IgD), immunoglobulin E (IgE), immunoglobulin G (IgG), or immunoglobulin M (IgM) antibodies IgE, IgG, or IgM antibodies. Preferred antibodies to detect are IgA, IgG and IgM antibodies.

Any animal that possesses maternal antibodies or generates antibodies in response to an infectious agent or corresponding vaccine can be tested in accordance with the present invention. In one embodiment, a preferred animal to test is an animal that was vaccinated (i.e., administered a vaccine) at least about six months, one year, two years, or three years prior to testing. In another embodiment, a preferred animal to test is an animal for whom infection or vaccination status is unknown. Suitable animals for whom to determine an immune status include, but are not limited to, cats (i.e., felids), dogs (i.e., canids), horses (i.e., equids), humans and other primates, ferrets and other Mustelids, cattle, sheep, swine, and rodents, as well as other companion animals (i.e., pets), food animals, work animals, or zoo animals. Preferred animals to test include cats, dogs, horses and other companion animals, with cats, dogs and horses being even more preferred. As used herein, a cat refers to any member of the cat family (i.e., Felidae), including domestic cats, wild cats and zoo cats. Examples of cats include, but are not limited to, domestic cats, lions, tigers, leopards, panthers, cougars, bobcats, lynx, jaguars, cheetahs, and servals. A preferred cat to test is a domestic cat. As used herein, a dog refers to any member of the family Canidae, including, but not limited to, domestic dogs, wild dogs, foxes, wolves, jackals, and coyotes and other members of the family Canidae. As used herein, a horse refers to an equid. An equid is a hoofed mammal and includes, but is not limited to, domestic horses and wild horses, such as, horses, assess, donkeys, and zebras. Preferred horses to test include domestic horses, including race horses.

A biological specimen refers to any sample that can be collected (i.e. obtained) from an animal in which antibodies may be found. A suitable biological specimen includes, but is not limited to, a bodily fluid composition or a cellular composition. Examples of a bodily fluid include, but are not limited to, blood, serum, plasma, saliva, urine, tears, aqueous humor, cerebrospinal fluid, lymph, nasal secretion, tracheobronchial aspirate, milk, colostrum, intestinal secretion, and feces, with blood, serum, plasma, saliva, urine, tears, milk and colostrum being preferred and blood, serum or plasma being even more preferred.

As used herein, the term contacting refers to combining or mixing, in this case, a biological specimen and a recombinant antigen of the present invention. Formation of a complex, or immunocomplex, between a recombinant antigen and any antibody selective for an infectious agent (i.e., an anti-infectious agent antibody) present in the biological specimen refers to the ability of the recombinant antigen to selectively bind to the antibody in order to form a stable complex that can be detected. As used herein, the term selectively binds to an antibody or specific for an antibody refers to the ability of a recombinant antigen of the present invention to preferentially bind to an antibody that indicates that the animal is protected from disease, without being able to substantially bind to other, unrelated, antibodies. Binding between the recombinant antigen and anti-infectious agent antibody is effected under conditions suitable to form a complex; such conditions (e.g., appropriate concentrations, buffers, temperatures, reaction times) as well as methods to optimize such conditions are known to those skilled in the art, and examples are disclosed herein. Examples of complex formation conditions are also disclosed in, for example, in Sambrook et al., ibid., and Harlow, et al., 1988, Antibodies, a Laboratory Manual, Cold Spring Harbor Labs Press; Harlow et al., ibid., in incorporated herein by reference in its entirety.

As used herein, the phrase detecting the presence or absence of a complex refers to determining if any complex is formed, i.e., assaying for the presence (i.e., existence) or absence (i.e., non-existence) of a complex. If complexes are formed, the amount of complexes formed can, but need not be, determined. Complex formation, or selective binding, between a recombinant antigen and anti-infectious agent antibody can be measured (i.e., detected, determined) using a variety of methods standard in the art; see, for example, Sambrook, et al., ibid., Harlow, et al., ibid., and examples herein.

A complex can be measured in a variety of ways including, but not limited to, one of the following assays: an enzyme-linked immunoassay, a radioimmunoassay, a fluorescence immunoassay, a luminescence assay (such as a cherni-luminescent assay or a bioluminescent assay), a phosphorescence assay, an immunoblot assay (e.g., a Western blot), an immunodot assay, an immunoprecipitation assay, a lateral flow assay, a flow-through assay, an agglutination assay, a particulate-based assay (e.g., using particulates such as, but not limited to, magnetic particles or plastic polymers, such as latex or polystyrene beads), and an electronic sensory assay (e.g., using an electronic chip). In one embodiment, it is preferred not to use a virus neutralization assay, a hemagglutination assay, or a complement fixation assay. Such assays are well known to those skilled in the art; see for, example, Harlow, et al., ibid. Assays can be used to give qualitative or quantitative results depending on how they are used.

Some assays, such as agglutination, particulate separation, and immunoprecipitation, can be observed visually (e.g., either by eye or by a machines, such as a densitometer or spectrophotometer) without the need for a detectable marker. In other assays, conjugation (i.e., attachment, joining) of a detectable marker to a recombinant antigen of the present invention or to an antibody-binding partner of the present invention that selectively binds to the antibody being detected aids in measuring complex formation. Conjugation is conducted in such a manner that the ability of a recombinant antigen or antibody-binding partner to selectively bind to anti-infectious agent antibodies is not compromised. Conjugation can be accomplished, for example, by joining a detectable marker to a recombinant antigen or antibody-binding partner or by constructing a genetic chimera that encodes a recombinant antigen fused to a detectable marker or an antibody-binding partner fused to a detectable marker.

Examples of detectable markers include, but are not limited to, an enzyme, a radioactive label, a fluorescent label, a luminescent label (e.g., a bio-luminescent label or a chemi-luminescent label), a chromophoric (e.g., colorimetric) label, a metal sol label, a metal-binding label, a physical label, an electronic label, or a ligand. A ligand refers to a molecule that binds selectively to another molecule. Preferred detectable markers include, but are not limited to, a phosphatase (e.g., alkaline phosphatase), a peroxidase (e.g., horseradish peroxidase), a beta-galactosidase, a luciferase, fluorescein, a radioisotope, a bead (e.g., a color bead, a magnetic bead), colloidal gold, biotin, avidin, and biotin-related compounds or avidin-related compounds (e.g., streptavidin or IMMUNOPURE® NeutrAvidin).

An antibody-binding partner of the present invention is any compound that can bind to an anti-infectious agent antibody of the present invention. Preferably an antibody-binding partner binds to the constant region of such an antibody, such as to the Fc region of an IgA, IgD, IgE, IgG, or IgM antibody. Examples of such antibody-binding partners include anti-isotype antibodies (e.g., anti-IgA antibodies, anti-IgD antibodies, anti-IgE antibodies, anti-IgG antibodies, and anti-IgM antibodies) that selectively bind to the constant region of antibodies of the animal being tested, antibody Fc receptors (e.g., IgA receptors, Igd receptors, IgE receptors, IgG receptors, IgM receptors), antibody-binding bacterial surface proteins (e.g., Protein A or Protein G, or recombinant forms of these proteins), antibody-binding cells (e.g., a B cell, T cell, or a macrophage), other antibody-binding eukaryotic cell surface proteins, and antibody-binding complement proteins, as well as any portion of these proteins that selectively bind to an anti-infectious agent antibody. Preferred antibody-binding partners include Protein A, Protein G, an anti-IgG antibody, an anti-IgM antibody, an anti-IgA antibody, an anti-IgE antibody, an Fc_{γ} receptor molecule, an Fc_{ε} receptor molecule, an Fc_{μ} receptor molecule, and an Fc_{α} receptor molecule as well as any portion of any of such proteins that selectively bind to the constant region of an anti-infectious agent antibody. It is within the scope of the present invention that a complex between an anti-infectious agent antibody and a recombinant antigen of the present invention can be determined using one or more layers and/or types of secondary antibodies or other binding compounds. For example, an unlabeled secondary antibody can be bound to an anti-infectious agent antibody and the unlabeled secondary antibody can then be bound by a labeled tertiary antibody.

In one embodiment of the present invention, the presence or absence of a complex is detected by applying a detection reagent that binds to the complex, if present, to obtain a test signal. The term applying refers to adding a detection reagent to the biological specimen after the recombinant antigen is combined with the specimen under conditions to form a complex with any anti-infectious agent antibody in the specimen. The detection reagent binds to any complex present and such binding results in a test signal, i.e., an event that can be detected. If anti-infectious agent antibody is present in the biological specimen, a test signal will ensue. If there is no anti-infectious agent antibody present, no test signal will occur. Preferably, the detection reagent comprises an antibody-binding partner of the present invention conjugated to a detectable marker of the present invention.

In one embodiment a complex can be formed and measured in solution. In another embodiment, a recombinant antigen of the present invention or an antibody-binding partner of the present invention can be immobilized on (e.g., coated onto) a substrate. Preferably, a recombinant antigen of the present invention is immobilized on a substrate. Immobilization techniques are known to those skilled in the art. Suitable substrates on which to immobilize a recombinant antigen or antibody-binding partner of the present invention or a composition include, but are not limited to, plastic, glass, gel, celluloid, paper, fabric, electronic chip, and particulate materials such as latex, polystyrene, nylon, nitrocellulose, agarose, cotton, PVDF (poly-vinylidene-fluoride), and magnetic resin. Suitable substrates include, but are not limited to, a well (e.g., microtiter dish well), a plate, a dipstick, a strip, a bead, a sponge, a lateral flow apparatus, a membrane, a filter, a tube, a dish, a celluloid-type matrix, a magnetic particle, an electronic sensory device (e.g., an electronic sensory chip), and other particulates. In one embodiment, a substrate, such as a particulate, can include a detectable marker.

In a preferred embodiment, a method to determine the immune status of an animal can be conducted within about one day, more preferably within about two hours, more preferably within about one hour, and even more preferably within a time period of between about one minute and about fifteen minutes.

A method of the present invention to detect immune status can be qualitative, quantitative, or semi-quantitative. In one embodiment, the method includes a step of comparing the intensity of a test signal of the present invention with a reference signal obtained by contacting a reference reagent with the detection reagent to determine the amount of anti-infectious agent antibody in the biological specimen. In one embodiment, the reference signal represents a threshold, such that if the test signal is more intense than the reference signal the animal from which the biological specimen is collected is deemed to be protected from infection by the infectious agent. In one embodiment the reference reagent is immobilized on a substrate, preferably on the same substrate as is a recombinant antigen. Suitable reference reagents include antibodies isolated from the same species of animal as is being tested. Preferred reference reagents to use in immune status assays for cats, dogs and horses, include feline antibodies, canine antibodies and equine antibodies, respectively.

One embodiment of a method of the present invention to determine the immune status of an animal is to determine the immune status with respect to more than one infectious agent. It is contemplated that any number of recombinant antigens can be used in such a determination. In one embodiment, a biological specimen from an animal is contacted with a recombinant calicivirus antigen, a recombinant herpesvirus antigen and a recombinant parvovirus antigen under conditions such that the immune status of the animal to calicivirus, herpesvirus and parvovirus infection is determined.

Another embodiment of the present invention includes the use of an immune status assay to determine whether a human should be treated for rabies virus infection. In such an embodiment, a biological specimen is collected from an animal suspected of having exposed the human to rabies virus infection and contacted with a recombinant rabies virus antigen in accordance with the present invention. Presence of a complex indicates that the human should be treated for rabies infection.

A preferred method to detect anti-infectious agent antibodies is an immunosorbent assay. In one embodiment, a recombinant antigen of the present invention is immobilized on a substrate, such as a microtiter dish well or a dipstick. A biological specimen collected from an animal is applied to the substrate and incubated under conditions sufficient to allow for complex formation. Excess fluid, if any, is removed and a detection reagent that can selectively bind to the anti-infectious agent antibody is added to the substrate and incubated to allow formation of a complex between the detection reagent and the recombinant antigen:anti-infectious agent antibody complex. Excess detection reagent is removed, a developing agent is added if required, and the substrate is submitted to a detection device for analysis. Alternatively, an antibody-binding partner as described above is immobilized on a substrate, and a biological specimen is incubated with the antibody-binding partner to form a complex. Complex detection can then be accomplished by applying a detectable marker-conjugated recombinant antigen of the present invention to the complex.

Another preferred method to determine the immune status of an animal is a lateral flow assay, examples of which are disclosed in U.S. Pat. No. 5,424,193, issued Jun. 13, 1995, by Pronovost et al.; U.S. Pat. No. 5,415,994, issued May 16, 1995, by Imrich et al; WO 94/29696, published Dec. 22, 1994, by Miller et al.; and WO 94/01775, published Jan. 20, 1994, by Pawlak et al.; each of these patent publications is incorporated by reference herein in its entirety. Another preferred method to determine the immune status of an animal is a flow-through assay, examples of which are disclosed in U.S. Pat. No. 4,632,901, issued Dec. 30, 1986 by Valkirs et al., and U.S. Pat. No. 4,727,019, issued Feb. 23, 1988, by Valkirs et al; U.S. Pat. No. 4,632,901, ibid., and U.S. Pat. No. 4,727,019, ibid., are both incorporated by reference herein in their entireties.

Another embodiment of the present invention is a method to determine whether to vaccinate an animal. Such a method includes the steps of: (a) contacting a biological specimen of the animal with a recombinant infectious agent antigen that is specific for detecting an antibody selective for that infectious agent, under conditions suitable for formation of a complex between the recombinant antigen and the antibody; and (b) detecting the presence or absence of the complex. Presence of such a complex indicates that the animal need not be vaccinated, whereas absence of such a complex indicates that the animal should be vaccinated. Detection of such a complex can be accomplished in a manner similar to that disclosed herein for determining the immune status of an animal.

Yet another embodiment of the present invention is an assay, or kit, to determine the immune status of an anima and/or to determine whether to vaccinate an animal. Such an assay includes (a) a recombinant infectious agent antigen that is specific for detecting an antibody selective for that infectious agent; and (b) a means to detect an antibody that selectively binds to the recombinant antigen. In one embodiment, the means includes a detection reagent of the present invention. An assay of the present invention can also, but need not, include (a) a solid support comprising a test area and a reference area; and (b) a reference reagent. Preferably the test area includes one or more recombinant antigens of the present invention and the reference area comprises one or more reference reagents of the present invention. An assay of the present invention can also, but need not, include a control area for assay validation. Preferably, a recombinant infectious agent antigen of the present invention is immobilized on a substrate such as those disclosed herein. Particularly preferred assays are ELISAs, lateral flow assays, and flow-through assays.

The following examples are provided for the purposes of illustration and are not intended to limit the scope of the present invention.

### EXAMPLES

It is to be noted that the Examples include a number of molecular biology, microbiology, innunology and biochemistry techniques considered to be known to those skilled in the art. Disclosure of such techniques can be found, for example, in Sambrook et al., ibid., Harlow et al., ibid., and related references.

### Example 1

This Example demonstrates that use of a whole virus preparation to determine the immune status of an animal leads to false positives and, as such, is an unacceptable reagent.

A. Purification of feline calicivirus and feline rhinotracheitis virus Feline rhinotracheitis virus (also known as feline herpesvirus, or FHV) and feline calicivirus (FCV) were cultured in Crandall Reese Feline Kidney (CRFK) cells in DMEM high glucose (available from Gibco BRL, Gaithersburg, Md.) with 2% fetal bovine serum (FBS) for FHV and no fetal bovine serum for FCV. Aliquots of titered (TCID₅₀) virus-containing tissue culture supernatant were collected and stored at −70° C. until use.

FCV- or FHV-containing supernatant aliquots were each thawed quickly in a 37° C. water bath and clarified by centrifugation at 1000×g for 10 min at 4° C. Five volumes of a 60% (w/v) Iodixanol solution (available from OptiPrepä, Nycomed, Oslo, Norway) were mixed with one volume of 0.8% NaCl, 60 mM HEPES, pH 7.4 to produce a 50% Iodixanol solution. Three ml of the Iodixanol-containing supernatant aliquot were transferred to 16×102 mm Beckman Ultra Clear centrifuge tubes (available from Beckman, Fullerton, Calif.). Three ml of the 50% Iodixanol solution were underlayed under the supernatant aliquot. The virus was sedimented by centrifugation at 100,000×g for 1 hr at 4° C. using a Beckman SW28 fixed-angle rotor (available from Beckman). The virus formed a sharp band on top of the Iodixanol cushion. Three ml of the supernatant were removed. The residual content of the tube was mixed to produce a concentrated virus suspension in approximately 25% Iodixanol. The suspension was transferred to 16×76 mm Beckman Quickseal tubes (available from Beckman). The residual air space in the heat seal tubes was filled with the 0.8% NaCl, 60 mM HEPES buffer and the tubes heat sealed. The tubes were centrifuged at 350,000×g for 1 to 3 hr at 4° C. using a Beckman VTi-65.1 rotor (available from Beckman). The rotor was allowed to decelerate from 21×g (500 rpm) without the brake. The seals on the tubes were ruptured, and most of the supernatant was removed with a long Pasteur pipette. Approximately 1 ml of fluid was left in each tube. This material was transferred to a common tube and the original tube was rinsed with 0.5 ml of 0.8% NaCl/60 mM HEPES buffer and that material was added to the common tube. Total protein was determined by the BioRad Protein Assay (available from BioRad, Richmond, Calif.). Aliquots of virus were stored at −70° C. Preparation purity was determined by ELISA. FCV purified in this manner is referred to as an Optiprep-purified FCV preparation, or Optiprep-purified FCV. FHV purified in this manner is referred to as an Optiprep-purified FHV preparation, or Optiprep-purified FHV.

B. Purification of feline panleukopenia virus Feline panleukopenia virus (FPV) was cultured in Crandall Reese Feline Kidney (CRFK) cells in DMEM high glucose with 2% fetal bovine serum. Aliquots of titered (TCID₅₀) virus-containing tissue culture supernatant were collected and stored at −70° C. until use.

A FPV-containing supernatant aliquot was clarified by centrifugation at 7000×g for 15 min at 4° C. The pellet was discarded and virus was precipitated from the supernatant by the addition of solid polyethylene glycol (PEG) 3350 to 0.75 M PEG, and 0.2 M sodium chloride. The mixture was incubated 30 min on ice and then centrifuged at 7000×g for 30 min at 4° C. The pellet was resuspended in 0.2 M boric acid buffer (pH 7.4) with 0.5M NaCl. The material was centrifuged at 450×g for 5 min to remove insoluble matter. The virus was banded in an isopyknic cesium chloride (CsCl) gradient (1.40 g/ml) by equilibrium centrifugation at 150,000×g for 20 hr at 4° C. (40,000 rpm in Beckman SW65 Ti rotor). Total protein was determined by the BioRad Protein Assay. Aliquots of virus were stored at −70° C. Preparation purity was determined by ELISA. FPV purified in this manner is referred to as a CsCl-purified FPV preparation, or CsCl-purified FPV.

C. Testing of a Whole FCV Preparation as an Immune Status Reagent

A Optiprep-purified FCV preparation, produced as described in Example 1A, as well as a preparation prepared in the same manner but in which CRFK cells were not infected with FCV (i.e., an Optiprep-purified non-infected cell, or NIC, preparation) was each tested for its ability to react with serum from FCV-vaccinated (positive) cats or barrier control (negative) cats by ELISA.

The ELISA was conducted as follows. The Optiprep-purified FCV and NIC preparations were each diluted according to protein concentration as indicated in Table 1 into 50 mM carbonate/bicarbonate buffer (pH 9.6). After dilution, plates were coated with a 100-μL aliquot of each dilution in wells in a PolySorp strip (Nunc, available from VWR Scientific, West Chester, Pa.). Each strip was placed in a strip holder plate and incubated overnight at 4° C. The coated wells were washed four times with PBST (10 mM PBS, containing 8.5 g NaCl, 0.20 g KH₂PO₄, and 1.16 g Na₂HPO₄ in 1 L water, at pH=7.2, 0.05% TWEEN® 20 (C₅₈H₁₁₄O₂₆; FW=1227, available from Fisher Scientific, Pittsburgh, Pa.), using an automatic plate washer (available from Bio-tek Instruments, Inc., Winooski, Vt.). After washing, a 200-μL aliquot of StabilCoat (available from SurModics, Eden Prairie, Minn.) was added to each well and the strips were incubated for one hour at 22° C. The wells were then washed four times with PBST using an automatic plate washer. Vaccinated (positive) or barrier (negative) cat serum was diluted 1:50 prior to addition to the wells with diluent A (PBST, 4% FBS, 0.5% ProClin 300 (available from Supelco, Bellefonte, Pa.). A 100-μL aliquot of the appropriate diluted serum was then added to each of the appropriate wells, and the plate was incubated for two hours at 22° C., followed by four washes with PBST using an automatic plate washer. Goat anti-cat IgG (H & L)-HRP (available from Kirkegaard & Perry Laboratories, Gaithersburg, Md.) was diluted in diluent A to 500 ng/ml, and a 100-μL aliquot was then added to each well. The plates were incubated for one hour at 22° C., followed by four washes with PBST using an automatic plate washer. A 100-μL aliquot of two-component substrate (TMB Peroxidase Substrate System, available from Kirkegaard & Perry Laboratories) was added to each of wells, which were then incubated at 22° C. for 5 min. Reactions were stopped by adding 100 μL of 1 M H₃PO₄ to each of the wells, at which time an automatic plate reader was used to determine O.D at 450 nm (using, for example, Molecular Devices SPECTRAMAX® 250, available from Molecular Devices, Sunnyvale, Calif.). ELISA results are shown in Table 1.

TABLE 1
ELISA using Optiprep-purified FCV or NIC to test serum collected from
FCV-vaccinated (positive) or barrier (negative) cats

| protein | positive | | | |
|---|---|---|---|---|
| (ng/ml) | (FCV) | negative (FCV) | positive (NIC) | negative (NIC) |
| 20000 | 4.15 | 0.61 | | |
| 10000 | 4.15 | 0.70 | 3.368 | 0.616 |
| 5000 | 4.15 | 0.88 | 3.231 | 0.506 |
| 2500 | 4.15 | 0.84 | 2.901 | 0.396 |
| 1250 | 4.15 | 0.86 | 2.485 | 0.362 |
| 625 | 4.15 | 0.74 | 2.035 | 0.303 |
| 313 | 4.04 | 0.66 | 1.586 | 0.264 |
| 156 | 4.00 | 0.62 | 1.204 | 0.244 |
| 78 | 3.72 | 0.52 | 0.782 | 0.216 |
| 39 | 3.22 | 0.45 | 0.721 | 0.165 |
| 20 | 2.68 | 0.36 | 0.629 | 0.134 |
| 10 | 2.34 | 0.36 | 0.598 | 0.124 |
| 5 | 2.16 | 0.31 | 0.653 | 0.13 |

These data indicate that although an Optiprep-purified FCV preparation can detect antibodies in FCV-vaccinated cats, so does an Optiprep-purified NIC preparation (i.e., a preparation produced from uninfected cells using a similar procedure). As such, whole FCV is an unacceptable reagent for the determination of the immune status of a cat due to the possibility of a high percentage of false positive reactions due to the presence of cellular proteins that react with serum from vaccinated cats.

D. Testing of a Whole FHV Preparation as an Immune Status Reagent

A Optiprep-purified FHV preparation, produced as described in Example 1A, as well as a preparation prepared in the same manner but in which CRFK cells were not infected with FHV (i.e., an Optiprep-purified non-infected cell, or NIC, preparation) was each tested for its ability to react with serum from FHV-vaccinated (positive) cats or barrier control (negative) cats by ELISA.

The ELISA was conducted as described in Example 1C except that an Optiprep-purified FHV preparation was used instead of an Optiprep-purified FCV preparation, serum from FHV-vaccinated cats was used, and preparation dilutions were conducted as indicated in Table 2. Results are shown in Table 2.

TABLE 2
ELISA using Optiprep-purified FHV or NIC to test serum collected from
FHV-vaccinated (positive) or barrier (negative) cats

| protein | positive | | | |
|---|---|---|---|---|
| (ng/ml) | (FHV) | negative (FHV) | positive (NIC) | negative (NIC) |
| 20000 | 4.13 | 0.02 | | |
| 10000 | 4.13 | 0.02 | 3.368 | 0.616 |
| 5000 | 4.15 | 0.00 | 3.231 | 0.506 |
| 2500 | 4.14 | 0.01 | 2.901 | 0.396 |
| 1250 | 4.01 | 0.13 | 2.485 | 0.362 |
| 625 | 3.73 | 0.37 | 2.035 | 0.303 |
| 313 | 3.47 | 0.46 | 1.586 | 0.264 |
| 156 | 2.95 | 0.40 | 1.204 | 0.244 |
| 78 | 2.25 | 0.47 | 0.782 | 0.216 |
| 39 | 1.68 | 0.38 | 0.721 | 0.165 |
| 20 | 1.40 | 0.50 | 0.629 | 0.134 |
| 10 | 0.80 | 0.26 | 0.598 | 0.124 |
| 5 | 0.72 | 0.17 | 0.653 | 0.13 |

These data indicate that although an Optiprep-purified FHV preparation can detect antibodies in FHV-vaccinated cats, so does an Optiprep-purified NIC preparation (i.e., a preparation produced from uninfected cells using a similar procedure). As such, whole FHV is an unacceptable reagent for the determination of the immune status of a cat due to the possibility of a high percentage of false positive reactions due to the presence of cellular proteins that react with serum from vaccinated cats.

E. Testing of a Whole FPV Preparation as an Immune Status Reagent

A CsCl-purified FPV preparation, produced as described in Example 1B, as well as a preparation prepared in the same manner but in which CRFK cells were not infected with FPV (i.e., a CsCl-purified non-infected cell, or NIC, preparation) was each tested for its ability to react with serum from FPV-vaccinated (positive) cats or barrier control (negative) cats by ELISA.

The ELISA was conducted as described in Example 1C except that a CsCl-purified FPV preparation was used instead of an Optiprep-purified FCV preparation, serum from FPV-vaccinated cats was used, and preparation dilutions were conducted as indicated in Table 3. Results are shown in Table 3.

TABLE 3
ELISA using CsCl-purified FPV or NIC to test serum collected from
FPV-vaccinated (positive) or barrier (negative) cats

| protein | positive | | | |
|---|---|---|---|---|
| (ng/ml) | (FPV) | negative (FPV) | positive (NIC) | negative (NIC) |
| 10000 | 4.082 | 0.468 | 3.368 | 0.616 |
| 5000 | 4.031 | 0.474 | 3.231 | 0.506 |
| 2500 | 3.947 | 0.492 | 2.901 | 0.396 |
| 1250 | 3.799 | 0.5 | 2.485 | 0.362 |
| 625 | 3.233 | 0.481 | 2.035 | 0.303 |
| 313 | 2.58 | 0.393 | 1.586 | 0.264 |
| 156 | 1.929 | 0.287 | 1.204 | 0.244 |
| 78 | 1.115 | 0.21 | 0.782 | 0.216 |
| 39 | 0.836 | 0.16 | 0.721 | 0.165 |
| 20 | 0.655 | 0.134 | 0.629 | 0.134 |
| 10 | 0.752 | 0.111 | 0.598 | 0.124 |
| 5 | 0.527 | 0.103 | 0.653 | 0.13 |

These data indicate that although a CsCl-purified FPV preparation can detect antibodies in FPV-vaccinated cats, so does a CsCl-purified NIC preparation (i.e., a preparation produced from uninfected cells using a similar procedure). As such, whole FPV is an unacceptable reagent for the determination of the immune status of a cat due to the possibility of a high percentage of false positive reactions due to the presence of cellular proteins that react with serum ftom vaccinated cats.

### Example 2

This Example describes the isolation and expression of nucleic acid molecules of the present invention that encode feline calicivirus coat proteins (FCVCPs) of the present invention. Also described is the purification of recombinant feline calicivirus coat proteins (rFCVCPs) of the present invention.

A. A nucleic acid molecule of 2016 nucleotides designated herein as nFCVCP₂₀₁₃ with a coding strand represented by SEQ ID NO:1, encoding a full-length FCVCP, was produced by PCR amplification and TA CLONING® using standard techniques, such as those described in Sambrook et al., ibid. Nucleic acid molecule nFCVCP₂₀₁₃ was ligated to recombinant vector λP_{R}cro/T² ori/RSET-B, described in PCT Publication No. WO 98/12563, published Mar. 26, 1998, by Grieve et al., in such a manner that the nucleotides of the recombinant vector encoding the N-terminal histidine (His) tag were ligated in frame with the nucleotides encoding the feline calicivirus coat protein. The resulting recombinant molecule, designated herein as pλP_{R}His-nFCVCP₂₀₁₃, was transformed into Escherichia coli to produce recombinant cell E. coli:pλP_{R}His-nFCVCP₂₀₁₃ using standard techniques, such as those disclosed in Sambrook et al., ibid. Recombinant cell E. coli:pλP_{R}His-nFCVCP₂₀₁₃ was cultured as described in WO 98/12563, ibid., to produce a 672-amino acid FCVCP protein, having SEQ ID NO:2, designated PFCVCP₆₇₁, fused to a His tag. The fusion protein, referred to herein as PHis-PFCVCP₆₇₁, was purified from E. coli by standard protein purification techniques.

B. A nucleic acid molecule of 1644 nucleotides, designated herein as nFCVCP₁₆₄₁ with a coding strand represented by SEQ ID NO:3, which spans nucleotides 373 to 2016 of SEQ ID NO: 1, encoding a mature FCVCP, was produced by PCR amplification and TA CLONING® as described in Example 2A. Nucleic acid molecule nFCVCP₁₆₄₁ was ligated to recombinant vector λP_{R}Cro/T² ori/RSET-B/Hisless, a modified version of recombinant vector λP_{R} cro/T² ori/RSET-B (described in Example 2A) from which codons encoding the His tag had been removed. The resulting recombinant molecule, designated herein as pλP_{R}-nFCVCP₁₆₄₁, was transformed into Escherichia coli to produce recombinant cell E. coli:pλP_{R}-nFCVCP₁₆₄₁ as described in Example 2A. Recombinant cell E. coli:pλP_{R}-nFCVCP₁₆₄₁ was cultured as described in Example 2A WO 98/12563, ibid., to produce a 548-amino acid FCVCP protein, designated PFCVCP₅₄₇, the amino acid sequence of which is represented herein as SEQ ID NO:4. PFCVCP₅₄₇ was purified from E. coli by standard protein purification techniques.

### Example 3

This Example describes the isolation and expression of nucleic acid molecules of the present invention that encode feline parvovirus capsid proteins (FPVVPs) of the present invention. Also described is the purification of recombinant feline parvovirus capsid proteins (rFPVVPs) of the present invention.

A. A nucleic acid molecule of 1755 nucleotides, designated herein as nFPVVP2₁₇₅₂ with a coding strand represented by SEQ ID NO:5, encoding a full-length feline parvovirus VP2 capsid protein, was produced by PCR amplification and TA CLONING® as described in Example 2A. Nucleic acid molecule nFPVVP2₁₇₅₂ was ligated to recombinant vector λP_{R}cro/T² ori/RSET-B as described in Example 2A to produce recombinant molecule pλP_{R}His-nFPVVP2₁₇₅₂, which was then transformed into Escherichia coli to produce recombinant cell E. coli:pλP_{R}His-nFPVVP2₁₇₅₂ as described in Example 2A. Recombinant cell E. coli:pλP_{R}His-nFPVVP2₁₇₅₂ was cultured as described in Example 2A to produce a 585-amino acid FPVVP2 protein, having SEQ ID NO:6, designated PFPVVP2₅₈₄, fused to a His tag. The fusion protein, referred to herein as PHis-PFPVVP2₅₈₄, was purified from E. coli by standard protein purification techniques.

B. A nucleic acid molecule of 729 nucleotides, designated herein as nFPVVP2C₇₂₉ with a coding strand represented by SEQ ID NO:7, which spans nucleotides 703 to 1431 of SEQ ID NO:5, encoding a truncated VP2 capsid protein, was produced by PCR amplification and TA CLONING® as described in Example 2A. Nucleic acid molecule nFPVVP2C₇₂₉ was ligated to recombinant vector λP_{R}cro/T² ori/RSET-B as described in Example 2A to produce recombinant molecule pλP_{R}His-nFPVVP2C₇₂₉, which was then transformed into Escherichia coli to produce recombinant cell E. coli:pλP_{R}His-nFPVVP2C₇₂₉ as described in Example 2A. Recombinant cell E. coli:pλP_{R}His-nFPVVP2C₇₂₉ was cultured as described in Example 2A to produce a 243-amino acid FPVVP2 protein, having SEQ ID NO:8, designated PFPVVP2C₂₄₃, fused to a His tag. The fusion protein, referred to herein as PHis-PFPVVP2C₂₄₃, was purified from E. coli by standard protein purification techniques.

Nucleic acid molecule nFPVVP2C₇₂₉ was also ligated to recombinant vector λP_{R}cro/T² ori/RSET-B/Hisless as described in Example 2B to produce recombinant molecule pλP_{R}-nFPVVP2C₇₂₉, which was then transformed into Escherichia coli to produce recombinant cell E. coli:pλP_{R}-nFPVVP2C₇₂₉ as described in Example 2A. Recombinant cell E. coli:pλP_{R}-nFPVVP2C₇₂₉ was cultured as described in Example 2A to produce a 243-amino acid FPVVP2 protein, designated herein as PFPVVP2C₂₄₃, the amino acid sequence of which is represented herein as SEQ ID NO:8. PFPVVP2C₂₄₃ was purified from E. coli by standard protein purification techniques.

C. A nucleic acid molecule of 1860 nucleotides, designated herein as nFPVpVP12₁₈₆₀ with a coding strand represented by SEQ ID NO:9, encoding a truncated VP1-VP2 capsid protein, was produced by PCR amplification and TA CLONING® as described in Example 2A. Nucleic acid molecule nFPVpVP12₁₈₆₀ was ligated to recombinant vector λP_{R}cro/T² ori/RSET-B as described in Example 2A to produce recombinant molecule pλP_{R}His-nFPVpVP12₁₈₆₀, which was then transformed into Escherichia coli to produce recombinant cell E. coli:pλP_{R}His-nFPVpVP12₁₈₆₀ as described in Example 2A. Recombinant cell E. coli:pλP_{R}His-nFPVpVP12₁₈₆₀ was cultured as described in Example 2A to produce a 620-amino acid FPVVP12 protein, having SEQ ID NO:10, designated PFPVpVP12₆₂₀, fused to a His tag. The fusion protein, referred to herein as PHis-PFPVpVP12₆₂₀, was purified from E. coli by standard protein purification techniques.

D. A nucleic acid molecule of 1431 nucleotides, designated herein as nFPVpVP2₁₄₃₁ with a coding strand represented by SEQ ID NO:11, which spans nucleotides 1 to 1431 of SEQ ID NO:5, encoding a truncated VP2 capsid protein, was produced by PCR amplification and TA CLONING® as described in Example 2A. Nucleic acid molecule nFPVpVP2₁₄₃₁ was ligated to recombinant vector λP_{R}cro/T² ori/RSET-B/Hisless as described in Example 2B to produce recombinant molecule pλP_{R}-nFPVpVP2₁₄₃₁, which was then transformed into Escherichia coli to produce recombinant cell E. coli:pλP_{R}-nFPVpVP2₁₄₃₁ as described in Example 2A. Recombinant cell E. coli:pλP_{R}-nFPVpVP2₁₄₃₁ was cultured as described in Example 2A to produce a 477-amino acid truncated FPVVP2 protein, designated PFPVpVP2₄₇₇, the amino acid sequence of which is represented as SEQ ID NO:12. PFPVpVP2₄₇₇ was purified fom E. coli by standard protein purification techniques.

Nucleic acid molecule nFPVpVP2₁₄₃₁ was also ligated to recombinant vector λP_{R}cro/T² ori/RSET-B as described in Example 2A to produce recombinant molecule pλP_{R}His-nFPVpVP2₁₄₃₁, which was then transformed into Escherichia coli to produce recombinant cell E. coli:pλP_{R}His-nFPVpVP2₁₄₃₁ as described in Example 2A. Recombinant cell E. coli:pλP_{R}His-nFPVpVP2₁₄₃₁ was cultured as described in Example 2A to produce a 477-amino acid truncated FPVVP2 protein, designated PFPVpVP2₄₇₇, with SEQ ID NO:12, fused to a His tag. The fusion protein, designated PHis-PFPVpVP2₄₇₇ was purified from E. coli by standard protein purification techniques.

### Example 4

This Example describes the isolation and expression of nucleic acid molecules of the present invention that encode feline herpesvirus glycoproteins of the present invention. Also described is the purification of recombinant feline herpesvirus glycoproteins (rFHVgB, rFHVgC, and rFHV gD proteins) of the present invention.

A. A nucleic acid molecule of 2832 nucleotides, designated herein as nFHVgB₂₈₂₉ with a coding strand represented by SEQ ID NO:13, encoding a full-length feline herpesvirus glycoprotein B protein, was produced by PCR amplification and TA CLONING® as described in Example 2A. Nucleic acid molecule nFHVgB₂₈₂₉ was ligated to recombinant vector λP_{R}cro/T² ori/RSET-B as described in Example 2A to produce recombinant molecule pλP_{R}His-nFHVgB₂₈₂₉, which was then transformed into Escherichia coli to produce recombinant cell E. coli:pλP_{R}His-nFHVgB₂₈₂₉ as described in Example 2A. Recombinant cell E. coli:pλP_{R}His-nFHVgB₂₈₂₉ was cultured as described in Example 2A to produce a 944-amino acid FHVgB protein, having SEQ ID NO: 14, designated PFHVgB₉₄₃, fused to a His tag. The fusion protein, referred to herein as PHis-PFHVgB₉₄₃, was purified from E. coli by standard protein purification techniques.

B. A nucleic acid molecule of 750 nucleotides, designated herein as nFHVgB₇₅₀ with a coding strand represented by SEQ ID NO:15, spanning nucleotides 1 to 750 of SEQ ID NO:13, encoding a truncated feline herpesvirus glycoprotein B protein, was produced by PCR amplification and TA CLONING® as described in Example 2A. Nucleic acid molecule nFHVgB₇₅₀ was ligated to recombinant vector λP_{R}cro/T² ori/RSET-B as described in Example 2A to produce recombinant molecule pλP_{R}His-nFHVgB₇₅₀, which was then transformed into Escherichia coli to produce recombinant cell E. coli:pλP_{R}His-nFHVgB₇₅₀ as described in Example 2A. Recombinant cell E. coli:pλP_{R}His-nFHVgB₇₅₀ was cultured as described in Example 2A to produce a 250-amino acid FHVgB protein, having SEQ ID NO:16, designated PFHVgB₂₅₀, fused to a His tag. The fusion protein, referred to herein as PHis-PFHVgB₂₅₀, was purified from E. coli by standard protein purification techniques.

C. A nucleic acid molecule of 1605 nucleotides, designated herein as nFHVgC₁₆₀₂ with a coding strand represented by SEQ ID NO:17, encoding a full-length feline herpesvirus glycoprotein C protein, was produced by PCR amplification and TA CLONING® as described in Example 2A. Nucleic acid molecule nFHVgC602 was ligated to recombinant vector λP_{R}cro/T² ori/RSET-B as described in Example 2A to produce recombinant molecule pλP_{R}His-nFHVgC₁₆₀₂, which was then transformed into Escherichia coli to produce recombinant cell E. coli:pλP_{R}His-nFHVgC₁₆₀₂ as described in Example 2A. Recombinant cell E. coli:pλP_{R}His-nFHVgC₁₆₀₂ was cultured as described in Example 2A to produce a 535-amino acid FHVGC protein, having SEQ ID NO:18, designated PFHVgC₅₃₄, fused to a His tag. The fusion protein, referred to herein as PHis-PFHVgC₁₋₃₄, was purified from E. coli by standard protein purification techniques.

D. A nucleic acid molecule of 1401 nucleotides, designated herein as nFHVgC₁₄₀₁ with a coding strand represented by SEQ ID NO:19, spanning nucleotides 97 to 1497 of SEQ ID NO:17, encoding a truncated feline herpesvirus glycoprotein C protein was produced by PCR amplification and TA CLONINGT as described in Example 2A. Nucleic acid molecule nFHVgC₁₄₀₁ was ligated to recombinant vector λP_{R}cro/T² ori/RSET-B as described in Example 2A to produce recombinant molecule pλP_{R}His-nFHVgC₁₄₀₁, which was then transformed into Escherichia coli to produce recombinant cell E. coli:pλP_{R}His-nFHVgC₁₄₀₁ as described in Example 2A. Recombinant cell E. coli:pλP_{R}His-nFHVgC₁₄₀₁ was cultured as described in Example 2A to produce a 467-amino acid FHVgC protein, having SEQ ID NO:20, designated PFHVgC₄₆₇, fused to a His tag. The fusion protein, referred to herein as PHis-PFHVgC₄₆₇, was purified from E. coli by standard protein purification techniques.

E. A nucleic acid molecule of 1401 nucleotides, designated nFHVgC₁₄₀₁₍ₒₚₜ₎, encoding feline herpesvirus protein PFHVgC₄₆₇ but in which a number of codons were optimized for expression in E. coli was produced as follows. A series of PCR mutagenesis steps was performed on nFHVgC₁₄₀₁, the coding strand of which is represented by SEQ ID NO:19, using standard techniques, such as those described in Sambrook et al., ibid., to target the following codons: two arginine codons spanning nucleotides 119 to 124 of SEQ ID NO:19; three serine codons spanning nucleotides 133 to 141 of SEQ ID NO:19; a glycine codon spanning nucleotides 724 to 726 of SEQ ID NO:19; and a leucine codon spanning nucleotides 727 to 729 of SEQ ID NO:19. The resulting nucleic acid molecule, namely nFHVgC₁₄₀₁₍ₒₚₜ₎, has a coding strand sequence as represented in SEQ ID NO:21. Nucleic acid molecule nFHVgC₁₄₀₁₍ₒₚₜ₎ was ligated to recombinant vector λP_{R}cro/T² ori/RSET-B/Hisless as described in Example 2B to produce recombinant molecule pλP_{R}-nFHVgC₁₄₀₁₍ₒₚₜ₎, which was then transformed into Escherichia coli to produce recombinant cell E. coli:pλP_{R}-nFHVgC₁₄₀₁₍ₒₚₜ₎ was described in Example 2A. Recombinant cell E. coli:pλP_{R}-nFHVgC₁₄₀₁₍ₒₚₜ₎ was cultured as described in Example 2A to produce a 467-amino acid FHVgC protein, designated PFHVgC₄₆₇₍ₒₚₜ₎. PFHVgC₄₆₇₍ₒₚₜ₎, the amino acid sequence of which is represented as SEQ ID NO:22, which is identical to SEQ ID NO:20, was purified from E. coli by standard protein purification techniques.

F. A nucleic acid molecule of 1125 nucleotides, designated herein as nFHVgD₁₁₂₂ with a coding strand represented by SEQ ID NO:23, encoding a full-length feline herpesvirus glycoprotein D protein, was produced by PCR amplification and TA CLONING® as described in Example 2A. Nucleic acid molecule nFHVgD₁₁₂₂ was ligated to recombinant vector λP_{R}cro/T² ori/RSET-B as described in Example 2A to produce recombinant molecule pλP_{R}His-nFHVgD₁₁₂₂, which was then transformed into Escherichia coli to produce recombinant cell E. coli:pλP_{R}His-nFHVgD₁₁₂₂ as described in Example 2A. Recombinant cell E. coli:pλP_{R}His-nFHVgD₁₁₂₂ was cultured as described in Example 2A to produce a 375-amino acid FHVgD protein, having SEQ ID NO:24, designated PFHVgD₃₇₄, fused to a His tag. The fusion protein, referred to herein as PHis-PFHVgD₃₇₄, was purified from E. coli by standard protein purification techniques.

G. A nucleic acid molecule of 900 nucleotides, designated herein as nFHVgD₉₀₀ with a coding strand represented by SEQ ID NO:25, spanning nucleotides 85 to 894 of SEQ ID NO:23, encoding a truncated feline herpesvirus glycoprotein D protein, was produced by PCR amplification and TA CLONING® as described in Example 2A. Nucleic acid molecule nFHVgD₉₀₀ was ligated to recombinant vector λP_{R}cro/T² ori/RSET-B as described in Example 2A to produce recombinant molecule pλP_{R}His-nFHVgD₉₀₀, which was then transformed into Escherichia coli to produce recombinant cell E. coli:pλP_{R}His-nFHVgD₉₀₀ as described in Example 2A. Recombinant cell E. coli:pλP_{R}His-nFHVgD₉₀₀ was cultured as described in Example 2A to produce a 300-amino acid FHVgD protein, having SEQ ID NO:26, designated PFHVgD₃₀₀, fused to a His tag. The fusion protein, referred to herein as PHis-PFHVgD₃₀₀, was purified from E. coli by standard protein purification techniques.

### Example 5

This Example describes the isolation and expression of nucleic acid molecules of the present invention that encode feline leukemia virus (FeLV) proteins of the present invention. Also described is the purification of recombinant feline herpesvirus proteins (rFeLVp27 and rFeLVgp70 proteins) of the present invention.

A. A nucleic acid molecule of 789 nucleotides, designated herein as nFeLVp27₇₅₉ with a coding strand represented by SEQ ID NO:27, encoding a mature FeLV p27 protein, was produced by PCR amplification and TA CLONING® as described in Example 2A. Nucleic acid molecule nFeLVp27₇₅₉ was ligated to recombinant vector λP_{R}cro/T² ori/RSET-B/Hisless as described in Example 2B to produce recombinant molecule pλP_{R}-nFeLVp27₇₅₉, which was then transformed into Escherichia coli to produce recombinant cell E. coli:pλP_{R}-nFeLVp27₇₅₉ as described in Example 2A. Recombinant cell E. coli:pλP_{R}-nFeLVp27₇₅₉ was cultured as described in Example 2A to produce a 263-amino acid FeLV p27 protein designated PFeLVp27₂₅₃, the amino acid sequence of which is represented as SEQ ID NO:28. PFeLVp27₂₅₃ was purified from E. coli by standard protein purification techniques.

B. A nucleic acid molecule of 1857 nucleotides, designated herein as nFeLVgp70₁₈₃₀ with a coding strand represented by SEQ ID NO:29, encoding a mature FeLV envelope glycoprotein 70 (gp70) protein, was produced by PCR amplification and TA CLONING® as described in Example 2A. Nucleic acid molecule nFeLVgp70₁₈₃₀ was ligated to recombinant vector λP_{R}cro/T² ori/RSET-B as described in Example 2A to produce recombinant molecule pλP_{R}His-nFeLVp27₁₈₅₇, which was then transformed into Escherichia coli to produce recombinant cell E. coli:pλP_{R}His-nFeLVp27₁₈₅₇ as described in Example 2A. Recombinant cell E. coli:pλP_{R}His-nFeLVp27₁₈₅₇ was cultured as described in Example 2A to produce a 619-amino acid FeLV gp70 protein designated PFeLVgp70₆₁₀, the amino acid sequence of which is represented as SEQ ID NO:30, fused to a His tag. The fusion protein, referred to herein as PHis-PFeLVgp70₆₁₀, was purified from E. coli by standard protein purification techniques.

C. A nucleic acid molecule of 1833 nucleotides, designated herein as nFeLVp27-gp70₁₈₃₃ with a coding strand represented by SEQ ID NO:31, encoding a fusion protein of the carboxy-terminus of FeLV Pr65-gag and gp70, was produced by PCR amplification and TA CLONING® as described in Example 2A. Nucleic acid molecule nFeLVp27-gp70₁₈₃₃ was ligated to recombinant vector λP_{R}cro/T² ori/RSET-B/Hisless as described in Example 2B to produce recombinant molecule pλP_{R}-nFeLVp27-gp70₁₈₃₃ which was then transformed into Escherichia coli to produce recombinant cell E. Coli:pλP_{R}-nFeLVp27-gp70₁₈₃₃ as described in Example 2A. Recombinant cell E. coli:pλP_{R}-nFeLVp27-gp70₁₈₃₃ was cultured as described in Example 2A to produce a 611-amino acid fusion protein, designated as PFeLVp27-gp70₆₁₁, the amino acid sequence of which is represented as SEQ ID NO:32. PFeLVp27-gp70₆₁₁ was purified from E. coli by standard protein purification techniques.

Nucleic acid molecule nFeLVp27-gp70₁₈₃₃ was also ligated to recombinant vector λP_{R}cro/T² ori/RSET-B as described in Example 2B to produce recombinant molecule pλP_{R}His-nFeLVp27-gp70₁₈₃₃ which was then transformed into Escherichia coli to produce recombinant cell E. coli:pλP_{R}His-nFeLVp27-gp70₁₈₃₃ as described in Example 2A. Recombinant cell E. coli:pλP_{R}His-nFeLVp27-gp70₁₈₃₃ was cultured as described in Example 2A to produce a 611-amino acid fusion protein, designated as PFeLVp27-gp70₆₁₁, the amino acid sequence of which is represented as SEQ ID NO:32, fused to a His tag. The fusion protein, designated PHis-PFeLVp27-gp70₆₁₁, was purified from E. coli by standard protein purification techniques.

### Example 6

This Example describes the isolation and expression of nucleic acid molecules of the present invention that encode canine distemper virus (CDV) proteins of the present invention. Also described is the purification of recombinant CDV hernagglutinin (rCDVH) and fusion (rCDVF) proteins of the present invention.

A. A nucleic acid molecule of 1812 nucleotides, designated herein as nCDVH₁₈₁₂ with a coding strand represented by SEQ ID NO:33, encoding a CDV hemagglutinin protein, was produced by PCR amplification and TA CLONING® as described in Example 2A. Nucleic acid molecule nCDVH₁₈₁₂ was ligated to recombinant vector λP_{R}cro/T² ori/RSET-B as described in Example 2A to produce recombinant molecule pλP_{R}His-nCDVHIS₁₈₁₂, which was then transformed into Escherichia coli to produce recombinant cell E. coli:pλP_{R}His-nCDVH₁₈₁₂ as described in Example 2A. Recombinant cell E. coli:pλP_{R}His-nCDVH₁₈₁₂ was cultured as described in Example 2A to produce a 604-amino acid protein designated PCDVH₆₀₄, the amino acid sequence of which is represented as SEQ ID NO:34, fused to a His tag. The fusion protein, designated PHis-PCDVH₆₀₄, was purified from E. coli by standard protein purification techniques.

B. A nucleic acid molecule of 1986 nucleotides, designated herein as nCDVF₁₉₈₆ with a coding strand represented by SEQ ID NO:35, encoding a CDV fusion protein, was produced by PCR amplification and TA CLONING® as described in Example 2A. Nucleic acid molecule nCDVF₁₉₈₆ was ligated to recombinant vector λP_{R}cro/T² ori/RSET-B as described in Example 2A to produce recombinant molecule pλP_{R}His-nCDVF₁₉₈₆, which was then transformed into Escherichia coli to produce recombinant cell E. coli:pλP_{R}His-nCDVFl₉₈₆ as described in Example 2A. Recombinant cell E. coli:pλP_{R}His-nCDVF₁₉₈₆ was cultured as described in Example 2A to produce a 662-amino acid protein designated PCDVF₆₆₂, the amino acid sequence of which is represented as SEQ ID NO:36, fused to a His tag. The fusion protein, designated PHis-PCDVF₆₆₂, was purified from E. coli by standard protein purification techniques.

### Example 7

This Example demonstrates an immune status assay of the present invention. In particular, this Example demonstrates a correlation between humoral immune responses in cats previously vaccinated with panleukopenia (FPV), herpesvirus 1 (FHV-1), and calicivirus (FCV) vaccines and protection of such cats from challenge infections.

Forty cats were treated in the following manner: 14 cats were vaccinated with FCV, FHV-1 and FPV vaccines once, 6 months prior to challenge; 12 cats were vaccinated with FCV, FHV-1 and FPV vaccines either once or twice, with the last vaccine given 30 to 36 months prior to challenge; and 14 cats were unvaccinated. Challenge was accomplished following USDA challenge protocols utilized for vaccine approval. An immune status ELISA was utilized to determine the amounts of anti-FCV antibodies, anti-FHV antibodies, and anti-FPV antibodies in the serum of each of the cats prior to challenge using, respectively, the following recombinant antigens of the present invention: recombinant FCV coat protein (rFCVCP) protein PFCVCP₅₄₇, the amino acid sequence of which is represented as SEQ ID NO:4, and the production of which is described in Example 2B; recombinant FHV glycoprotein C (rFHVgC) protein PHis-PFHVgC₄₆₇, a fusion protein of FHVgC₄₆₇, the amino acid sequence of which is represented by SEQ ID NO:22, the production of which is described in Example 4D; and recombinant FPV VP2 capsid protein (rFPVVP2) protein PFPVpVP2₄₇₇ the amino acid sequence of which is represented as SEQ ID NO:12, and the production of which is described in Example 3D. Cutoff values were based on results from 30 unvaccinated cats. ELISAs were conducted in a similar manner to those described in Example 1C, with the following modifications: The specified recombinant antigens were used to coat plates (100 μL per well) at the following concentrations: rFCVCP protein PFCVCP₅₄₇ (starting concentration of 3 mg/ml) was diluted to 20 ng/ml (1:150,000 dilution); rFHVgC protein PFHVgC₄₆₇ (starting concentration of 2.24 mg/ml) was diluted to 50 ng/ml (1:44,800); and rFPVVP2 protein PFPVpVP2₄₇₇ (starting concentration of 1.12 mg/ml) was diluted to 120 ng/ml (1:9333). For wells containing rFCVCP and rFHVgC antigens, cat serum being tested was diluted 1:800 in diluent A; for wells containing rFPVVP2 antigen, the cat serum being tested was diluted 1:100 with diluent A.

Antibody levels were compared to clinical scores (FCV, FHV-1) or development of neutropenia (FPV). Cats were considered protected against FCV or FHV-1 if the clinical score was ≦50% of the mean of the unvaccinated cat group clinical score. Correlations between anti-FCV, anti-FHV and anti-FPV antibody levels and respective clinical scores for FCV, clinical scores for FHV-1, and development of neutropenia (FPV) are shown, respectively in Tables 4, 5, and 6.

TABLE 4
Correlation between clinical scores after FCV challenge and anti-FCV
antibody levels measured by ELISA using recombinant antigen
PFCVCP₅₄₇

| Sample | Group | OD Ave | OD SD | ELISA | Clin Score |
|---|---|---|---|---|---|
| 79 | vaccine I | 4.200 | 0.000 | + | 0 |
| 80 | vaccine I | 4.200 | 0.000 | + | 1 |
| 93 | vaccine I | 4.200 | 0.000 | + | 5 |
| 100 | vaccine I | 4.200 | 0.000 | + | 3 |
| 116 | vaccine I | 4.200 | 0.000 | + | 0 |
| 118 | vaccine I | 4.200 | 0.000 | + | 4 |
| 119 | vaccine I | 4.200 | 0.000 | + | 1 |
| 122 | vaccine I | 4.200 | 0.000 | + | 0 |
| 123 | vaccine I | 4.200 | 0.000 | + | 1 |
| 130 | vaccine I | 4.200 | 0.000 | + | 8 |
| 148 | vaccine I | 4.200 | 0.000 | + | 0 |
| 155 | vaccine I | 4.200 | 0.000 | + | 2 |
| 156 | vaccine I | 4.200 | 0.000 | + | 0 |
| 7029 | vaccine I | 4.200 | 0.000 | + | 0 |
| QVY3 | vaccine II | 4.200 | 0.000 | + | 2 |
| AMI4 | vaccine II | 4.200 | 0.000 | + | 0 |
| AMX1 | vaccine II | 4.200 | 0.000 | + | 0 |
| G444 | vaccine II | 4.200 | 0.000 | + | 0 |
| BWN3 | vaccine II | 4.200 | 0.000 | + | 0 |
| QWM3 | vaccine II | 4.200 | 0.000 | + | 0 |
| QVF3 | vaccine II | 4.200 | 0.000 | + | 0 |
| G087 | vaccine II | 4.200 | 0.000 | + | 0 |
| 3592 | vaccine II | 4.200 | 0.000 | + | 0 |
| 1959 | vaccine II | 4.200 | 0.000 | + | 2 |
| AME5 | vaccine II | 4.200 | 0.000 | + | 0 |
| 3513 | vaccine II | 4.200 | 0.000 | + | 0 |
| 7086 | control I | | | − | 7 |
| 7090 | control I | | | − | 17 |
| 7113 | control I | | | − | 19 |
| 7115 | control I | | | − | 23 |
| 7122 | control I | | | − | 12 |
| 7123 | control I | | | − | 27 |
| 7124 | control I | | | − | 24 |
| 7131 | control I | | | − | 21 |
| 7132 | control I | | | − | 34 |
| 7133 | control I | | | − | 25 |
| ALV3 | control II | | | − | 44 |
| ALT2 | control II | | | − | 35 |
| ALV5 | control II | | | − | 38 |
| ALZ1 | control II | | | − | 47 |
| AIY2 | negative | 0.447 | 0.213 | − | |
| AIW5 | negative | 0.383 | 0.098 | − | |
| AIY3 | negative | 0.514 | 0.255 | − | |
| AIU5 | negative | 0.479 | 0.206 | − | |
| AIW7 | negative | 0.463 | 0.094 | − | |
| AIY2 | negative | 0.345 | 0.090 | − | |
| AIU4 | negative | 0.440 | 0.118 | − | |
| AIW6 | negative | 0.389 | 0.071 | − | |
| AIV1 | negative | 0.427 | 0.111 | − | |
| AIW1 | negative | 0.307 | 0.098 | − | |
| AIW3 | negative | 0.299 | 0.104 | − | |
| AIU3 | negative | 0.389 | 0.041 | − | |
| AIW4 | negative | 0.368 | 0.197 | − | |
| AIW2 | negative | 0.429 | 0.181 | − | |
| AIY1 | negative | 2.370 | 1.125 | + | |
| | Neg. Ave | 0.406 | | | |
| | Neg. SD | 0.064 | | | |
| | Ave + 2SD | 0.533 | | | |

TABLE 5
Correlation between clinical scores after FHV-1 challenge and
anti-FHV antibody levels measured by ELISA using recombinant
antigen PFHVgC₄₆₇

| Sample | Group | OD Ave | OD SD | ELISA | Clin Score |
|---|---|---|---|---|---|
| 79 | vaccine I | 0.612 | 0.238 | +/− | 1 |
| 80 | vaccine I | 0.823 | 0.219 | + | 12 |
| 93 | vaccine I | 0.412 | 0.152 | − | 38 |
| 100 | vaccine I | 1.203 | 0.087 | + | 2 |
| 116 | vaccine I | 0.776 | 0.165 | + | 5 |
| 118 | vaccine I | 3.064 | 0.405 | + | 1 |
| 119 | vaccine I | 0.697 | 0.047 | + | 5 |
| 122 | vaccine I | 0.702 | 0.148 | + | 7 |
| 123 | vaccine I | 0.929 | 0.134 | + | 4 |
| 130 | vaccine I | 1.291 | 0.352 | + | 14 |
| 148 | vaccine I | 0.769 | 0.297 | + | 6 |
| 155 | vaccine I | 3.659 | 0.473 | + | 3 |
| 156 | vaccine I | 3.563 | 0.212 | + | 1 |
| 7029 | vaccine I | 0.460 | 0.080 | − | 42 |
| 3512 | vaccine II | 0.285 | 0.109 | − | 10 |
| 3514 | vaccine II | 1.764 | 0.596 | + | 8 |
| 3515 | vaccine II | 0.663 | 0.239 | + | 8 |
| 3519 | vaccine II | 1.349 | 0.389 | + | 14 |
| 3522 | vaccine II | 0.575 | 0.178 | − | 11 |
| 3528 | vaccine II | 0.660 | 0.257 | + | 11 |
| 3530 | vaccine II | 0.922 | 0.205 | + | 13 |
| 3531 | vaccine II | 0.404 | 0.101 | − | 11 |
| 3532 | vaccine II | 0.708 | 0.294 | + | 8 |
| 3535 | vaccine II | 1.574 | 0.584 | + | 16 |
| 3537 | vaccine II | 2.761 | 0.338 | + | 9 |
| 3542 | vaccine II | 0.407 | 0.173 | − | 17 |
| 7086 | control I | 0.271 | 0.036 | − | 24 |
| 7090 | control I | 0.207 | 0.015 | − | 19 |
| 7113 | control I | 0.296 | 0.070 | − | 19 |
| 7115 | control I | 0.327 | 0.209 | − | 15 |
| 7122 | control I | 0.259 | 0.055 | − | 22 |
| 7123 | control I | 0.258 | 0.039 | − | 16 |
| 7124 | control I | 0.215 | 0.016 | − | 18 |
| 7131 | control I | 0.807 | 0.118 | + | 16 |
| 7132 | control I | 0.290 | 0.102 | − | 27 |
| 7133 | control I | 0.259 | 0.042 | − | 14 |
| 2110 | control II | 0.377 | 0.287 | − | 26 |
| 2112 | control II | 0.396 | 0.125 | − | 33 |
| 2116 | control II | 0.185 | 0.076 | − | 37 |
| 2119 | control II | 0.295 | 0.116 | − | 42 |
| AIY2 | negative | 0.208 | 0.036 | − | |
| AIW5 | negative | 0.271 | 0.128 | − | |
| AIY3 | negative | 0.402 | 0.031 | − | |
| AIU5 | negative | 0.192 | 0.008 | − | |
| AIY1 | negative | 0.222 | 0.024 | − | |
| AIW7 | negative | 0.310 | 0.021 | − | |
| AIY2 | negative | 0.240 | 0.038 | − | |
| AIU4 | negative | 0.402 | 0.158 | − | |
| AIW6 | negative | 0.199 | 0.049 | − | |
| AIV1 | negative | 0.374 | 0.056 | − | |
| AIW1 | negative | 0.233 | 0.045 | − | |
| AIW3 | negative | 0.283 | 0.045 | − | |
| AIU3 | negative | 0.175 | 0.046 | − | |
| AIW4 | negative | 0.164 | 0.057 | − | |
| AIW2 | negative | 0.323 | 0.070 | − | |
| | Neg. Ave | 0.266 | | | |
| | Neg. SD | 0.040 | | | |
| | Ave + 2SD | 0.346 | | | |

TABLE 6
Correlation between development of neutropenia after FPV
challenge and anti-FPV antibodies measured by ELISA using
recombinant antigen PFPVpVP2₄₇₇

| Sample | Group | OD Ave. | OD SD | ELISA | Panleuk? |
|---|---|---|---|---|---|
| 79 | vaccine I | 3.952 | 0.294 | + | no |
| 80 | vaccine I | 0.748 | 0.099 | + | no |
| 100 | vaccine I | 1.625 | 0.324 | + | no |
| 116 | vaccine I | 2.915 | 0.373 | + | no |
| 118 | vaccine I | 3.432 | 0.374 | + | no |
| 119 | vaccine I | 2.820 | 0.428 | + | no |
| 122 | vaccine I | 2.174 | 0.278 | + | no |
| 123 | vaccine I | 2.780 | 0.410 | + | no |
| 130 | vaccine I | 0.678 | 0.194 | + | no |
| 148 | vaccine I | 0.300 | 0.073 | − | no |
| 155 | vaccine I | 1.550 | 0.247 | + | no |
| 156 | vaccine I | 0.808 | 0.206 | + | no |
| 7029 | vaccine I | 1.041 | 0.136 | + | no |
| 3512 | vaccine II | 0.505 | 0.122 | − | no |
| 3514 | vaccine II | 0.450 | 0.074 | − | no |
| 3515 | vaccine II | 0.547 | 0.115 | − | no |
| 3519 | vaccine II | 1.675 | 0.214 | + | no |
| 3522 | vaccine II | 0.292 | 0.042 | − | no |
| 3528 | vaccine II | 0.395 | 0.091 | − | no |
| 3530 | vaccine II | 0.369 | 0.102 | − | no |
| 3531 | vaccine II | 0.534 | 0.155 | − | no |
| 3532 | vaccine II | 0.427 | 0.145 | − | no |
| 3535 | vaccine II | 0.345 | 0.078 | − | no |
| 3537 | vaccine II | 1.221 | 0.353 | + | no |
| 3542 | vaccine II | 0.377 | 0.061 | − | no |
| 7132 | control I | 1.115 | 0.297 | + | yes |
| 7086 | control I | 0.301 | 0.063 | − | yes |
| 7090 | control I | 0.262 | 0.012 | − | yes |
| 7113 | control I | 0.275 | 0.065 | − | yes |
| 7115 | control I | 0.596 | 0.157 | − | yes |
| 7122 | control I | 0.278 | 0.087 | − | yes |
| 7123 | control I | 0.378 | 0.213 | − | yes |
| 7124 | control I | 0.615 | 0.308 | +/− | yes |
| 7131 | control I | 0.377 | 0.083 | − | yes |
| 7133 | control I | 0.310 | 0.114 | − | yes |
| 2110 | control II | 0.299 | 0.071 | − | yes |
| 2112 | control II | 0.578 | 0.199 | − | yes |
| 2116 | control II | 0.324 | 0.125 | − | yes |
| 2119 | control II | 0.306 | 0.079 | − | yes |
| AIY2 | negative | 0.236 | 0.042 | − | |
| AIW5 | negative | 0.145 | 0.093 | − | |
| AIY3 | negative | 0.240 | 0.071 | − | |
| AIU5 | negative | 0.153 | 0.055 | − | |
| AIY1 | negative | 0.266 | 0.081 | − | |
| AIW7 | negative | 0.195 | 0.092 | − | |
| AIY2 | negative | 0.214 | 0.138 | − | |
| AIU4 | negative | 0.196 | 0.111 | − | |
| AIW6 | negative | 0.162 | 0.043 | − | |
| AIV1 | negative | 0.292 | 0.074 | − | |
| AIW1 | negative | 0.228 | 0.068 | − | |
| AIW3 | negative | 0.121 | 0.030 | − | |
| AIU3 | negative | 0.122 | 0.037 | − | |
| AIW4 | negative | 0.165 | 0.053 | − | |
| AIW2 | negative | 0.209 | 0.066 | − | |
| | Neg. Ave | 0.196 | | | |
| | Neg. SD | 0.030 | | | |
| | Ave + 2SD | 0.256 | | | |

These data indicate the utility of an immune status of the present invention in predicting that a cat is protected from viral challenge. Specifically, the results in Table 4 indicate that all 26 vaccinated cats were protected from FCV challenge and that each of those cats had antibody levels predicting protection. The results in Table 5 indicate that 22 of 26 vaccinated cats were protected from FHV-1 challenge and that 18 of the 22 protected cats had antibody levels predicting protection. Of the four cats in this group that were not protected, 2 cats had antibody levels predicting lack of protection and 2 cats had antibody levels predicting protection. The results in Table 6 indicate that neutropenia was detected in all 14 unvaccinated cats but in none of the vaccinated cats, confirming panleukopenia in the unvaccinated cats. Of the vaccinated cats, 14 of the 25 cats available for study had FPV antibody levels predicting protection.

In conclusion, an immune status assay of the present invention shows high positive correlation with protection from challenge in healthy, vaccinated cats exposed to virulent FCV, FHV-1, or FPV.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1-39. (canceled)

40. A method to vaccinate a susceptible animal against an infectious agent, said method comprising the steps of:
(a) contacting a biological specimen from said animal with a recombinant protein capable of forming a complex with an antibody specific for said infectious agent under conditions suitable for formation of said complex, wherein said recombinant protein is from said infectious agent; and
(b) detecting the presence or absence of said protein: antibody complex; and
(c) in the absence of said complex, vaccinating said animal against said infectious agent.

41. The method of claim 40, wherein said biological specimen is selected from the group consisting of blood, serum, plasma, saliva, urine, tears, aqueous humor, cerebrospinal fluid, lymph, nasal secretion, tracheobronchial aspirate, milk, colostrum, intestinal secretion and feces.

42. The method of claim 40, wherein said animal is selected from the group consisting of a cat, a dog and a horse.

43. The method of claim 40, wherein said detection step comprises performing an assay selected from the group consisting of an enzyme-linked immunoassay, a radioimmunoassay, a fluorescence immunoassay, a luminescence assay, a phosphorescence assay, an immunoblot assay, an immunodot assay, an immunoprecipitation assay, a lateral flow assay, a flow-through assay, an agglutination assay, a particulate-based assay, and an electronic sensory assay.

44. The method of claim 40, wherein said detection step comprises applying a detection reagent that binds to said complex, if present, to obtain a test signal, wherein the presence or absence of a test signal is indicative of the need to vaccinate said animal.

45. The method of claim 44, wherein said detection reagent comprises an antibody-binding partner conjugated to a detectable marker.

46. The method of claim 45, wherein said antibody-binding partner is selected from the group consisting of an Fc-binding antibody, an Fc receptor, and an antibody-binding bacterial surface protein.

47. The method of claim 45, wherein said detectable marker is selected from the group consisting of an enzyme, a radioactive label, a fluorescent label, a luminescent label, a phosphorescent label, a chromophoric label, a metal sol label, a metal-binding label, a physical label, an electronic label, and a ligand.

48. The method of claim 40, wherein said recombinant protein is selected from the group consisting of a calicivirus protein, a distemper virus protein, a herpesvirus protein, a leukemia virus protein, a rabies virus, an adenovirus and a parvovirus protein.

49. The method of claim 40, wherein said recombinant protein is selected from the group consisting of a feline calicivirus capsid protein, a feline herpesvirus glycoprotein B protein, a feline herpesvirus glycoprotein C protein, a feline herpesvirus glycoprotein D protein, a feline parvovirus VP12 protein, a feline parvovirus VP2 protein, a feline leukemia virus p²7 protein, a feline leukemia virus gp70 protein, a feline leukemia virus p27-gp70 fusion protein, a canine distemper virus fusion protein, a canine adenovirus protein, and a canine distemper virus hemagglutinin protein.

50. The method of claim 40, wherein said recombinant protein comprises an amino acid sequence having at least 85% identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36.

51. The method of claim 40, wherein said recombinant protein comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36.

52. The method of claim 40, wherein said recombinant protein is encoded by a nucleic acid sequence having at least 85% identity with a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35.

53. The method of claim 40, wherein said recombinant protein is encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35.

54. A method to protect a previously vaccinated animal against infection by an infectious agent, said method comprising:
(a) obtaining a biological specimen from an animal that had been vaccinated at least six (6) months prior to obtaining said biological specimen;
(b) contacting said biological specimen with a recombinant protein capable of forming a complex with an antibody specific for said infectious agent under conditions suitable for formation of said complex, wherein said recombinant protein is from said infectious agent;
(c) determining if said complex is present; and
(d) in the absence of said complex, vaccinating said animal against said infectious agent.

55. The method of claim 54, wherein said recombinant protein is selected from the group consisting of a calicivirus protein, a distemper virus protein, a herpesvirus protein, a leukemia virus protein, a rabies virus, an adenovirus and a parvovirus protein.

56. The method of claim 54, wherein said recombinant protein is selected from the group consisting of a feline calicivirus capsid protein, a feline herpesvirus glycoprotein B protein, a feline herpesvirus glycoprotein C protein, a feline herpesvirus glycoprotein D protein, a feline parvovirus VP12 protein, a feline parvovirus VP2 protein, a feline leukemia virus p27 protein, a feline leukemia virus gp70 protein, a feline leukemia virus p27-gp70 fusion protein, a canine distemper virus fusion protein, a canine adenovirus protein, and a canine distemper virus hemagglutinin protein.

57. The method of claim 54, wherein said recombinant protein comprises an amino acid sequence having at least 85% identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36.

58. The method of claim 54, wherein said recombinant protein comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34 and SEQ ID NO:36.

59. A method to protect an animal against infection by an infectious agent, said method comprising the steps of:
(a) contacting a biological specimen of said animal with a recombinant protein capable of forming a complex with an antibody specific for said infectious agent under conditions suitable for formation of said complex;
(b) applying a detection reagent capable of binding to said complex to produce a test signal and a reference reagent to produce a reference signal;
(c) detecting the test signal and the reference signal; and
(d) comparing the intensity of the test signal with the intensity of the reference signal to determine the immune status of said animal and if the reference signal is more intense than the test signal, vaccinating the animal against the infectious agent.
